(19) United States
(12) Patent Application Publication
(10) Pub. No.: US 2003/0157715 A1
(43) Pub. Date: Aug. 21, 2003

(54) Modulation of chromosome function by chromatin remodeling agents

(76) Inventor: Ulrich Laemmli, Onex (CH)

Correspondence Address: Cooper & Dunham, LLP, 1185 Avenue of the Americans, New York, NY, 10036, US

(21) Appl. No.: 09/892,085

(22) Filed: Jun. 26, 2001

Related U.S. Application Data
(60) Provisional application No. 60/213,931, filed on Jun. 26, 2000.

(51) Int. Cl. C12N 15/87

(52) U.S. Cl. 435/455

(57) ABSTRACT

The present invention concerns a process for modulating the function of a DNA element in a eukaryotic cell, comprising the step of contacting a genomic DNA element, so-called “chromatin responsive element” (CRE), with a compound having a molecular weight of less than approximately 5 KDa, and having the capacity to bind in a_sequence-specific manner to said CRE, said step of contacting being carried out in conditions permitting chromatin remodeling of the CRE by said compound, wherein said chromatin remodeling of the CRE alters the activity of one or more other DNA elements, so called “modulated DNA elements” in the genome.

The present invention relates to a process for the modulation of chromosome function using sequence-specific chromatin remodeling agents. The invention also relates to chromatin remodeling agents which specificially target chromatin responsive elements in the genome, and to their use in modulating endogenous and heterologous gene function in a cell. The invention further relates to fluorescent chromatin remodeling agents, and their use in probing epigenetic status and location of DNA in nuclei, and their use for cytological/structural determinations, including quantitative estimations of specific DNA sequences in cells and chromosomal material.

In eukaryotic cells, DNA is folded first in the chromatin fiber and then into chromosomes by several hierarchical levels of organisation. This organisation changes dynamically around the cell cycle to facilitate different chromosomal functions. These structural levels of chromosomes are brought about by the evolutionarily conserved histones, various non-histone proteins and oligonucleotide protein complexes. The complex formed between DNA and these components is referred to as chromatin.

The first order of chromatin structure can be considered to be the “beads on a string” structure created when the DNA is wrapped around individual histones. Here the chromatin has the appearance of spherical particles connected by thin fibers. Each of these bead structures is known as a nucleosome, associated with approximately 200 base pairs of DNA. The nucleosome “beads” or “core particles” comprise highly protected DNA segments of 146 base pairs tightly wrapped around two each of histones H2A, H2B, H3 and H4 (the histone octamer). The stretch of 146 base pairs makes almost two full turns around the disc-shaped histone octamer. The remaining DNA provides the linker between adjacent nucleosomes. A single molecule of histone H1 is associated with each nucleosome, and serves to “seal” the two turns of DNA around the histone octamer.

Higher order chromatin structure involves the further assembly of the nucleosomes into filaments of 300 Angstroms, where the chromatin is proposed to be compacted by winding into a “solenoid”-type structure containing six nucleosomes per turn. Most chromatin is present in this form in interphase nuclei. Differences in the degree of folding, i.e. in the chromatin structure, in different regions of the chromosome play an important role in determining whether a particular gene is active in a particular cell. Indeed, chromatin status is one of the main upstream steps in gene regulation since it determines whether or not DNA-binding factors can gain access to the DNA. Chromatin state is thus an important element in epigenetic control of gene expression.

The chromatin fiber is thought to be partitioned into transcriptionally active (competent) and inactive domains. Experimentally these domains are revealed by their different sensitivity to digestion by enzymes such as DNase I, restriction enzymes or cleavage by topoisomerase II. Active chromatin (also called open) domains are generally more sensitive (more accessible) to digestion by these enzymes than inactive (less accessible) ones. At the global morphological level, it is possible to observe by light microscopy in nuclei two extreme structural (epigenetic) states of chromatin organisation. One is called heterochromatin and the other euchromatin. Heterochromatin generally reflects transcriptionally inactive (structurally compact) chromatin and euchromatin is generally enriched in transcriptionally active (competent), more open chromatin.

A growing number of activities have been described that mediate changes in chromatin structure (chromatin remodeling) that facilitate chromosome function by rendering the chromatin fiber more accessible to nuclear factors. The high molecular weight ATPase dependent activities include complexes such as SWI/SNF, a highly conserved 2 MDa multisub-unit assembly. The modifications brought about by such complexes include changes in the DNA conformation on the histone, alteration of histone conformation, and changes in histone/DNA interactions (Peterson C., and Workman J., Curr. Opinion in Genetics& Development, 2000, 10:187-192).

Chromatin remodeling not only gives rise to gene activation but can also lead to gene silencing. This affects both endogenous and heterologous DNA elements. The precise mechanisms involved in chromatin-mediated gene-silencing are as yet unclear, but may involve the facilitated binding of silencing factors (including enzymes), or the spreading of the heterochromatin-like states and/or chemical modifications. If the inserted gene is juxtaposed within or near such chromatin states, then gene silencing can occur. Furthermore, integration of multiple copies of heterologous genes can give rise to interactions between repeated sequences which in turn trigger the formation of inactive genetic states. Indeed, methylation induced by repeats can lead to chromatin modification.

Cis-acting DNA elements involved in chromatin remodeling have to date not been clearly identified, nor has their mechanism of action been elucidated.

Candidate elements are “Scaffold Associated Regions” (SARs) which are very AT-rich fragments several hundred base pairs in length composed of numerous clustered irregularly spaced runs of As and Ts (called A tracts) These DNA sequences specifically associate with the nuclear scaffold and possibly define the bases of chromatin loops. The possible involvement of SARs in higher order chromatin structure and gene expression has been suggested by a number of authors, although direct proof has not been obtained and the precise relationship between SAR function and nuclear organisation remains to be elucidated.

Data obtained in Drosophila using a high-affinity high-molecular weight SAR-binding protein called MATH20, expressed specifically in the larval eye imaginal discs (Girard et al., 1998). MATH20 was found to suppress the position effect variegation (PEV) phenotype manifested by the silencing of the white gene which, as a result of an inversion, is juxtaposed close to the heterochromatin of the X chromosome. On the basis of one current model, the authors suggested that MATH20 may be binding to a giant approximately 11 Mb reiterated SAR in the form of satellite III repeats, thereby disrupting the cooperative interaction of compacting proteins responsible for heterochromatin formation and transmission into the juxtaposed euchromatic region. According to this hypothesis, the binding of MATH20 would energetically disfavour the spreading of the polymerizing proteins into the surrounding euchromatic region, thus restoring the activity of the white gene.

An alternative, mutually non exclusive model has also been proposed according to which SAR function is mediated by sequences that readily unwind under torsional stress (Bode et al., Science 1992 255, 195-197).

The nature of the cis-DNA elements involved in chromatin remodeling, and their mechanism of action, has therefore not been unambiguously identified to date. The use of chromatin remodeling as a means of epigenetic control of gene function, and more generally of chromosome function, has therefore not been possible.

It is an object of the present invention to establish the existence and identity of DNA sequence motifs involved in chromatin remodeling.

It is also an object of the present invention to identify a means by which the chromatin state of these elements can be specifically remodelled and consequently by which specific regulation of chromosome function in cis or in trans can be effected.

It is a further object of the invention to provide chemical compounds which specifically interact with these elements and bring about chromatin remodeling to specifically regulate chromosome function.

It is also an object of this invention to establish that small DNA sequence-specific compounds binding to chromatin responsive elements (CRE) can mediate modeling.

The objectives of the present invention have been fulfilled by the identification, by the inventors, of chromatin responsive elements (CRE) in the genome. The inventors have also demonstrated that molecules capable of binding in a sequence-specific manner to the CREs trigger chromatin remodeling and thereby directly or indirectly regulate gene function in a predetermined genomic segment or segments.

Specifically, the invention concerns a process for modulating the function of a DNA element in a eukaryotic cell,

comprising the step of contacting a genomic DNA element, so-called “chromatin responsive element” (CRE),

with a compound having the capacity to bind in a sequence-specific manner to said CRE, and preferably having a molecular weight of less than approximately 5 KDa,

said step of contacting being carried out in conditions permitting chromatin remodeling of the CRE by said compound,

wherein said chromatin remodeling of the CRE alters the activity of one or more other DNA sequences in the genome.

The present inventors have thus shown that chromosome function is regulated in cis and in trans by DNA sequences, designated CREs, whose chromatin status affects the activity of other genomic sequences. They have also shown that the binding of sequence-specific compounds to the CREs causes chromatin remodeling of the CRE, and thereby elicit the regulatory action. Consequently chromosome function can be regulated by contacting the CREs with binding compounds.

In the context of the present invention, the term Chromatin Responsive Element (CRE) signifies a DNA sequence whose chromatin status allows the modulation of chromosome function in cis or in trans. The remodeling of the chromatin of the CRE, brought about by the DNA-binding molecule, causes a change in the function of (a) chromosome segment(s) different from, or including, the CRE.

In the context of the present invention, “chromatin remodeling” signifies any change in the chromatin, including changes to DNA conformation with respect to the histones such as: modification of the rotational phasing of the DNA on the histone octamer or modified accessibility of the DNA to DNA-binding proteins. The histone conformation may also be modified for example by rearranging or evicting components of the histone octamer, histone H1 or non-histone proteins. Changes in DNA/histone interactions may be made, for example by modifying the total length of DNA per nucleosome, by reducing or increasing nucleosome stability, or by modifying nucleosome mobility in cis or in trans.

The epigenetic state of the CRE is modified as a result of the chromatin remodeling. The epigenetic state of a DNA element can be considered to be the information content of the element which arises from characteristics other than its sequence.

The DNA element(s) whose function is modified in cis or in trans by the chromatin remodeling of the CRE will be referred to herein as “the modulated DNA element”. This element is DNA and associated proteins. It may also undergo an epigenetic alteration, including chemical modification such as methylation, as a result of the change in the CRE, for example the change in chromatin state of the CRE may give rise to a change in the chromatin state of the modulated DNA element, thus modifying its function. However, the modulation of function of this DNA element may ultimately arise from other types of changes such as redistribution, displacment, inhibition, enhancement of binding factors, initially caused by the chromatin remodeling of the CRE.

The CRE or the modulated DNA element(s) may comprise heterochromatin, heterochromatin-like DNA, euchromatin or naked DNA. According to a preferred embodiment the CRE is heterochromatin or heterochromatin-like and its remodeling converts it to a euchromatin-like accessible state.

The CRE may comprise single copy DNA or multicopy DNA, and it may contain identical or non-identical sequence motifs, or functionally interacting multipartite DNA segments. Particularly preferred CREs comprise repeat sequences such as satellite DNA, for example a series of GAGAA repeats. The CRE may comprise a DNA element involved in chromosome structure and function such as Scaffold Associated Regions (SARs), which are AT-rich fragments composed of numerous clustered, irregularly spaced runs of As and Ts. Alternatively, the CRE may comprise unwinding motifs, non-B type DNA structure (containing kinks or bends), or DNA elements with a propensity to position nucleosomes.

The CREs are usually, but not always, in non-coding, transcriptionally inactive sequences. The CRE may have a length ranging from about 6 to several thousand base-pairs. In the latter case, only part of the CRE is targeted by the sequence-specific DNA binding molecule. If the CRE encompasses repeat sequences, multiple binding molecules will bind within the CRE.

The modulated DNA element may be on the same DNA molecule as the CRE, in which case the CRE is said to be cis-acting. The modulatory effect can be exerted by the CRE in a local manner (i.e. over several tens of base pairs), or in a long distance manner (i.e. from about 100 upto several thousand base pairs), and can indeed extend over the whole of the chromosome. Thus the modulated DNA element may be positioned immediately flanking the CRE, or may be separated from the CRE by tens, hundreds or thousands of base pairs. An example of such a situation is where a heterologous gene has integrated into the genome in a position juxtaposing a heterochromatic satellite region. This embodiment of the invention is illustrated in the examples below by the white mottled PEV phenotype experiments.

The CRE and the modulated DNA element may coincide. In this case, the chromatin remodeling of the CRE gives rise to a direct effect on the function of the CRE-containing DNA element.

According to a further embodiment of the invention, the CRE may also be trans-acting in that the modulated DNA is or are not on the same DNA molecule as the CRE, or are not directly linked to the CRE. The modulatory effect exerted by chromatin remodeling of trans-acting CREs can arise as a result of displacement, redistribution, inhibition, or enhancement of DNA-binding factors which affect gene function. This embodiment of the invention is illustrated in the examples below by the brown-dominant PEV phenotype experiments.

According to the invention, the DNA element whose function is modulated by the CRE (i.e. the “modulated DNA element”) can be any potentially active or inactive DNA element. Particularly preferred DNA elements comprise regions involved in the binding of DNA-binding proteins, for example transcription regulatory regions, locus-control regions, origins of replication, boundary/insulation elements, chromosome structural elements.

The chromatin state of the modulated DNA element, prior to modulation, may be heterochromatic, heterochromatin-like, or euchromatic. It may also be naked DNA. After modulation mediated by the chromatin remodeling of the CRE, the chromatin state of the modulated DNA element may be changed or unchanged with respect to its state before modulation. Often, the modulation involves the conversion of the modulated DNA element from a heterochromatin-like state to a euchromatin-like state.

The modulatory effect of a given CRE is specific in so far as it is exerted on a particular DNA or series of DNA segments. Depending on the CRE in question, one unique DNA may be modulated, or on the contrary a multiplicity of DNA segments may be affected. Moreover, the effect exerted on the modulated DNA may in itself give rise to a cascade of further cis or trans modulatory effects. Thus the spectrum of effects which can be achieved using the present invention is broad and can be controlled by choice of the CRE, and of the CRE-binding molecule. Preferred CREs are unique in the genome.

The modulation induced by the chromatin remodeling of the CRE can involve one or more of the following effects: restoration of chromosome function, loss of chromosome function, enhancement of chromosome function, reduction of chromosome function, prevention of chromosome function, modification of the temporal or spatial specificity of gene function, and maintenance of chromosome function. Particularly preferred effects include restoration of gene function, for example by suppression of cis or trans epigentic gene silencing. This variant of the invention is particularly applicable for ensuring the function of heterologous genes, but can also be employed in therapy for activating endogenous genes which have become epigenetically silenced.

Another preferred embodiment is the loss of gene function by redistribution, displacement or inhibition of euchromatic binding factors involved in chromosome function, or by allowing the binding of such factors.

According to the invention, the CREs and the modulated DNA(s) may both be endogenous to the cell. In such a situation, the process of modulating chromosome function in accordance with the invention comprises simply the introduction into the cell of the sequence-specific CRE binding compound. This variant of the invention is particularly applicable for activating endogenous genes which are epigenetically silenced, either as a result of chromosome rearrangements or for example as a result of tissue and developmental specificity. It can also be used to induce loss of function of endogenous genes which are otherwise active.

In another variant, a CRE which is endogenous to the cell is used in combination with a modulated DNA which is heterologous to the cell. According to this variant, a heterologous DNA to be modulated is introduced into the cell in conditions allowing its integration in a chromosomal location where the endogenous CRE can exert its modulatory effect. Since the CREs of the invention can be chosen such as to exert their effect in cis over a short or long distance, or even in trans, it is possible to achieve the functional interaction between the endogenous CRE and the heterologous modulated DNA without undue effort. According to this variant, the sequence-specific CRE-binding compound is selected to bind to a CRE which has the capacity to functionally interact with the particular heterologous DNA of interest.

A further variant involves the use of a heterologous CRE in association with an endogenous modulated DNA. This variant of the invention involves introducing the heterologous CRE into the cell, in conditions allowing its integration in a chromosomal location where it can exert its modulatory effect on the endogenous gene in question. The CRE-binding compound must also be introduced. Again, the fact that the CREs of the invention can be chosen to exert their effect in cis over a short or long distance, or even in trans, allows the functional interaction between the heterologous CRE and the endogenous modulated DNA to be achieved without undue effort. This variant of the invention can be used for example to modulate the activity of chromosomal sequences which cannot be sufficiently regulated by endogenous CREs either as a result of the nature of the sequences involved, or as a result of positioning on the chromosome.

A further variant involves the use of a heterologous CRE in association with a heterologous modulated DNA. This variant of the invention is particularly useful for modifiying the epigenetic state of heterologous genes, for example for preventing or reducing epigenetic gene silencing. The process according to this variant of the invention comprises:

transforming a cell, preferably in a stable manner, with a nucleic acid sequence comprising the heterologous gene, and with a nucleic acid sequence comprising the heterologous CRE,

introducing into the cell a compound which has the capacity to bind in a sequence-specific manner to said heterologous CRE,

said step of contacting being carried out in conditions permitting chromatin remodeling of the heterologous CRE by said compound,

wherein said chromatin modelling of the CRE modulates the epigenetic state of the heterologous gene.

According to this variant, the heterologous CRE and the heterologous gene to be modulated may be introduced into the cell on the same or separate molecules of DNA, and they may be introduced simultaneously or subsequently one to the other. Furthermore, the introduction of the sequence-specific binding compound may be carried out prior to, simultaneously with, or subsequent to the introduction of the nucleic acids carrying the heterologous sequences of interest. In a preferred embodiment, the CRE and heterologous gene are introduced into the cell on the same molecule of DNA and the CRE-binding molecule is introduced subsequently when stable transformation has been established.

Again according to this embodiment, the heterologous CRE may act in cis or in trans on the heterologous gene.

Preferred examples of this variant of the invention include the use of satellite sequences as a CRE, in association with heterologous genes encoding growth factors, hormones, receptor proteins, viral proteins, regulatory RNAs, tumour suppressor genes, haemoglobin gens, genes involved in the immune response, therapeutic protein factors.

The process of the invention may be carried out in vivo, in vitro or ex vivo. In vivo use is particularly preferred. For ex vivo use, cells are taken from an organism and genetically modified to contain either a heterologous CRE or a hetereologous gene to be modulated, or both, and are reimplanted in the body. The sequence-specific CRE-binding molecule is introduced into the modified cells by any approriate method such as ingestion, injection, topical application etc.

For in vitro use, the three essential components of the invention, that is the CRE, the DNA whose function is to be modulated and the CRE-binding compound are combined in vitro in conditions allowing binding of the compound to the CRE.

According to the invention, the cell in which the chromatin-remodeling mediated modulation is effected can be eukaryotic or prokaryotic. Eukaryotic is particularly preferred. Suitable examples are vertebrate cells, invertebrate cells, plant cells, particularly mammalian cells, insect cells, or yeast cells. Human cells may be used. Cells from animal species useful in the production of heterologous proteins or in animal models, for example, bovine, ovine, avian, fish, equine, simian cells etc. are all suitable. Plant cells are also particularly preferred Indeed, heterologous genes inserted into plants are particularly susceptible to gene silencing and thus the technique of the invention is advantageous.

The preferred CREs of the invention are satellite sequences. However, the invention is not limited to such sequences. Further CREs can be identified using the teaching of the invention. Specifically, a series of compounds which specifically bind in the vicinity of the DNA element to be modulated are used, and any compound(s) affecting the epigenetic state e.g. facilitating the interaction of factors, is selected. Knowledge of the chromatin structure, for example the position of the nucleosomes and the DNA-binding factors (e.g. transcription factors), is useful to select candidate CRE motifs. Initially the epigenetic state of said DNA element to be modulated and the alteration thereof by compounds is monitored in vitro with the help of chromatin probes such as nucleases. This rapidly identifies compounds with the desired property. Subsequently, in vivo experiments are carried out to evaluate the phenotypical changes

The sequence-specific CRE binding compound may bind to the CRE through major groove interactions, minor-groove interactions, phosphate back-bone interactions, or a combination of these types of binding. According to a particularly preferred embodiment of the invention, the sequence-specific compound binds to the DNA minor groove.

The compound preferably has a molecular weight of less than 5 KDa, for example less than 4.5 kDA, or less than 4 kDa.

The sequence-specific CRE binding compound is preferably cell-permeable, greatly facilitating its introduction into the cell. For administration to animals, including administration to humans for therapeutic purposes, the molecules can thus be administered orally, topically, by injection etc.

In the context of the present invention, a molecule is said to bind in a sequence specific manner to the CRE target if the cell or organism in which the binding occurs presents no intolerable side-effects or toxicity as a result of the binding. By intolerable side effects or toxicity is meant life-threatening, or of sufficient gravity to cause undesired disruption of metabolism and biological function. Preferably, a molecule which binds in a sequence specific manner is capable of specifically recognising a DNA target sequence of at least 6, preferably at least 8, more preferably at least 10, even more preferably at least 12 and most preferably at least 18 nucleotides, in a chromatin context

The CRE-binding compounds of the invention preferably have an apparent binding affinity with respect to the CRE, of at least 5×10⁷ M⁻¹, as measured by in vitro techniques, such as footprinting techniques. More preferably the compound has an apparent binding affinity of at least 1×10⁹ M⁻¹, and even more preferably of at least 5×10¹⁰ M⁻¹.

Particularly preferred examples of CRE-binding compounds of the invention are DNA-binding organic oligomers comprising heterocycles, for example wherein the heterocycles have at least one annular nitrogen, oxygen or sulphur.

Examples of such oligomers include heterocycles chosen from pyrrole, imidazole, triazole, pyrazole, furan, thiazole, thiophene, oxazole, pyridine, or derivatives of any of these compounds wherein the ring NH group is substituted. Such compounds are described in Bailly C., and Chaires J., Bioconjugate Chem, Vol 9 No5, 1998, 513-538.

Particularly preferred compounds contain N-methylpyrrole (Py) and/or N-methylimidazole (Im), and may further contain aliphatic amino acids such as β-alanine and γ-aminobutyric acid. The synthesis of DNA-specific compounds of this type containing N-methylpyrrole (Py) and/or N-methylimidazole (Im), has been described (Geierstanger et al 1994). These pseudo-peptides, based on the structure of naturally occurring distamycin, bind DNA in the minor groove as antiparallel dimers (Pelton and Wemmer 1989). Their sequence-specificity depends on the side-by side pairing of this dimer where an Im opposite a Py (Im/Py) targets a GC base pair, a Py/Im recognizes a CG base pair and a Py/Py pair is degenerate for both AT or TA base pairs (White et al., 1997). Py-Im compounds have been shown to be cell permeable (Gottesfeld et al., 1997). These compounds will be referred to hereinafter as polyamides.

Using the pairing rules mentioned above, an appropriate CRE-polyamide is synthesised to recognise a given CRE. The sequence of the CRE determines the structure and composition of the polyamide. Many CRE-binding compounds can be made applying these rules.

A non-limiting example of a general formula which can be adapted to fit a particular CRE sequence is the following Formula I:

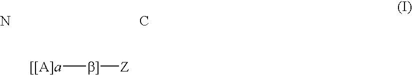

wherein

A is a monomer unit selected from the group consisting of an aromatic amino acid residue, particularly a heterocycle having at least one annular nitrogen, or the aliphatic amino acid β alanine (β), or fluorescent derivatives of said aromatic amino acid residues;

a represents an integer from 6 to 9,

β represents β-alanine

Z represents dimethylaminopropylamide (Dp) or another end group, or a fluorescent derivative thereof,

each solid line represents a covalent bond,

N and C indicate the N- and C-terminal extremities of the molecule, respectively,

with the proviso that:

the multiple A monomer units may be the same or different.

According to a preferred embodiment, in the above Formula I, a is 7 or 8.

According to a further embodiment, [A]a comprises at least four aromatic amino acids, and [A]a does not comprise a stretch of more than three contiguous aromatic amino acids.

Preferably, the multiple A units of Formula I comprise N-methylpyrrole (Py) and/or N-methylimidazole (Im) An example of this type of molecule has the formula (II):

[A₁]-[A₂]-[A₃]-[A₄]-[A₅]-[A₆]-[A₇]-β-Z (II)

wherein β and Z are as previously defined,

[A₄] is β,

[A₁] to [A₃], and [A₅] to [A₇] are chosen from N-methylpyrrole (Py) and/or N-methylimidazole (Im).

Preferred embodiments of Formula II are those wherein [A₁] to [A₃], and [A₅] to [A₇] are each N-methylpyrrole (Py)

Another preferred molecule has the formula (III):

[A₁]-[A₂]-[A₃]-[A₄]-[A₅]-[A₆]-[A₇]-[A₈]-β-Z (III)

wherein β and Z are as previously defined,

[A₁] to [A₈] are chosen from N-methylpyrrole (Py), N-methylimidazole (Im) and a β alanine residue, with the proviso that the [A] immediately adjacent to each Im on the N-terminal side is a β alanine residue.

A specific examples of the CRE-binding molecules of the invention are:

[Im-β-Im-Py-β-Im-β-Im-β]-Dp

The above molecules are well suited for binding to CREs since they show high affinity and specificity and are cell permeable. Further details are provided in the Examples.

The invention also relates to a gene expression kit suitable for modulating the epigenetic state of a heterologous gene in a cell, said kit comprising:

a nucleic acid molecule comprising said heterologous gene;

a nucleic acid molecule comprising a so-called heterologous “CRE”, said heterologous CRE being a sequence whose chromatin status allows the modulation of chromosome function in cis or trans;

a compound having a molecular weight of less than approximately 5 KDa, and having the capacity to bind in a sequence-specific manner to said CRE.

The heterologous CRE in such a kit may comprise a satellite sequence, for example a SAR-like AT tract or a GAGAA repeat sequence. Such kits may be used in gene therapy. The heterologous gene may be any gene of interest for example those cited earlier.

The invention also relates to a cell containing a compound, preferably having a molecular weight of less than 5 KDa, and having the capacity to bind in a sequence-specific manner to a genomic CRE, said CRE being a sequence whose chromatin status allows the modulation of chromosome function in cis or trans Preferably the compound within the cell binds the DNA-minor groove, although major groove binders and phosphate back-bone binders may also be contemplated.

The cell according to the invention may additionally contain

a nucleic acid molecule comprising a heterologous gene

a nucleic acid molecule comprising a so-called heterologous “CRE”, said heterologous CRE being a sequence whose chromatin status allows the modulation of chromosome function in cis or trans.

The cell of the invention may be a eukaryotic cell or a prokaryotic cell, as cited earlier.

Non-human organisms comprising such cells also form part of the invention. The organism may be a non-human animal. It may or may not be transgenic, depending upon whether any of the three components of the invention, i.e. the CRE, the modulated DNA element and the CRE-binding molecule are stably introduced into the cell by stable transformation.

Particularly preferred organisms are plants, which may be non-transgenic or transgenic. Gymnosperms and angiosperms are particularly suitable for use in the present invention, the latter group including monocotyledons and dicotyledons.

The invention also relates to a compound having the capacity to bind, in a sequence-specific manner, to a predetermined CRE, said CRE being a sequence whose chromatin status allows modulation of chromosome function in cis or in trans, with the proviso that said compound is not distamycin, HMG-I/Y, MATH20.

In a preferred embodiment, the compound has a molecular weight of less than 5 KDa and has the capacity to specifically recognise a sequence of at least 6 nucleotides. Even more preferably, the compound has the capacity to specifically recognise a sequence of at least 8, or at least 10 nucleotides, for example at least 15 or 16 nucleotides. The compound is preferably cell permeable.

Examples of such compounds are discussed earlier.

The compounds of the invention may be combined with a suitable physiologically acceptable excipient to prepare pharmaceutical compositions for use in humans or animals. Particularly preferred excipients are those for oral, topical, sub-cutaneous, intramuscular administration.

The pharmaceutical compositions of the invention may be used together with other pharmaceutical compositions comprising the necessary nucleic acid molecules for production of heterologous CREs and modulated elements. Such associations comprise a first pharmaceutical composition containing

a nucleic acid molecule comprising a heterologous gene; a nucleic acid molecule comprising a so-called heterologous “CRE”, said heterologous CRE being a sequence whose chromatin status allows the modulation of chromosome function in cis or trans, said nucleic acid molecules being in association with a physiologically acceptable excipient, and

a second pharmaceutical composition comprising a compound having the capacity to bind, in a sequence-specific manner, to said CRE, in association with a physiologically acceptable excipient.

The compounds, compositions associations of compositions and kits according to the invention can be used in therapy, for example in therapy of genetic disorders resulting from epigenetic status. Examples of such disorders are fragile X syndrome and imprinting disorders such as Wilm's tumour, and Prader-Willi syndrome.

The compounds and kits of the invention can also be used in a non-therapeutic manner for modulation of the expression of heterologus genes in cells, particularly eukaryotic cells. The cells may be in culture or in vivo. When the method is carried out in vivo, the organism may be transgenic or non-transgenic.

According to a particularly preferred embodiment, the compounds of the invention are fluorescent or fluorescently labelled. More particularly, this aspect relates to a DNA-binding compound capable of sequence specific binding to genomic DNA, said compound being an oligomer comprising cyclic heterocycles having at least one annular nitrogen, and optionally at least one aliphatic amino acid residue, wherein said compound is fluorescent or fluorescently labelled. The CRE-binding compounds described above are particularly preferred variants of this aspect of the invention.

It has surprisingly been shown by the present inventors that the addition of a fluorescent tag to the DNA-binding molecules does not alter the specificity of the binding. This permits the use of the fluorescent derivatives for cytological/structural determinations, including quantitative estimations of specific DNA sequences in cells and chromosomal material.

The fluorescent tags are usually added at the N or C terminal of the molecule, and can be a fluorescent dye such as fluorescein, dansyl, Texas red, isosulfan blue, ethyl red, malachite green, rhodamine and cyanine dyes.

The fluorescent CER-binding molecules can be used for probing the epigenetic state and location of DNA in chromosomes and nuclei, and for diagnosis of pathological conditions arising from epigenetic status, including pre-symptomatic diagnosis.

Further uses of the fluorescent derivatives of the invention are chromosome marking, diagnosis, forensic studies, and affiliation studies.

### FIGURE LEGENDS

Various aspects of the invention are illustrated in the figures:

FIG. 1: DNase I footprint assays with P9 and P7.

DNase I cleavage pattern in the presence of P9 and P7. Ligand concentrations are indicated at the top of each lane. The position of each of the AT-tracts is indicated by square brackets. Panel A shows the footprints of P9 and P7 on probe W9. This probe is composed of head-to-tail tandem repeats of an oligonucleotide with a 9 bp AT-tract.

FIG. 2: Staining of Drosophila nuclei with fluorescently tagged oligopyrroles.

Isolated Kc nuclei were stained with ethidium bromide and fluorescein-tagged oligopyrroles as indicated. Note that P9 (panel A) highlights as intense green foci satellites I and III and that the general nucleoplasmic background staining of P9 is low.

FIG. 3 Binding specificity of P31 and GAGA factor Panel A: DNAse I footprinting experiment with P31 and affinity cleavage with P31E are shown on GAF31 and the Brown I probes. The GAF31 and Brown I probes contains a (AAGAG)₂ motif and GAGA factor (GAF) binding site from the Ubx promoter (Biggin et al., 1988). Note that P31 does not bind the typical GAF binding (Ubx). The Brown I oligo (a tandem repeat) includes an (AAGAG)s binding site and a degenerate P31 binding site (AACAC)₂ as indicated. P31 concentrations used (nM) are indicated. Lanes labeled P31E (top) are affinity cleavage reactions with 1 nM of P31E on either probe. Binding orientations of P31E on these probes are indicated by arrowheads on the brackets pointing towards the N-terminus of the molecule. The letter G refers the G nucleotide cleavage reaction. Panel B: DNAse I footprinting experiment with purified GAGA factor (GAF) on the GAF31 probe. Note that GAF binds both the (AAGAG)2 motif and the binding site from the Ubx promoter.

FIG. 4: The fluorescent polyamide P31T specifically highlights the GAGAA satellite V

Isolated Kc nuclei and polytene chromosomes were stained with DAPI (blue), P31T (Texas red-labeled P31), P9F (Fluorescein tagged P9). Panel A: The green P9F foci are proposed to highlight satellites I and III. P31T marks the separate positions of the GAGAA satellites. Panels B & C: The black and white panels display the red and green channels of panel A, respectively. Panel D: Staining of brown-dominant polytene chromosome with DAPI, P31T and P9F. The polytene banding pattern is shown in blue (DAPI). P31T highlights in red the heterochromatic GAGAA repeats of the allele bwD at 59E.

FIG. 5: Oligopyrroles induced chromatin opening of satellite III.

Kc nuclei were incubated with mitotic Xenopus egg extracts in the presence of the various polyamides and then further treated with VM26 to accumulate the so-called cleavable complexes of topoisomerase II. Cleavage in Drosophila satellite III was revealed by southern blotting. Satellite III contains a major topoisomerase II cleavage site once per 359-bp repeat. The extent of the cleavage activity is reflected by the development of the ladder of multimers of the basic repeat. All panels included controls with (+) and lanes without Vm26 (−). The massive activation of cleavage (chromatin opening) mediated by P9 and the reduced activity P31 in this assay is shown.

FIG. 6: Binding Mode of Polyamide P9 and P31

Synthesis and characterization of these DNA satellite-specific polyamides is described in the examples. Both compounds, P9 and P31 were found to bind their targets with subnanomolar affinity in a 1:1 drug to DNA complex by hydrogen bonding schemes as proposed in this figure. Panel A: Proposed 1:1 binding model for the complex of P9 (Py-Py-Py-β-Py-Py-Py-β-Dp) where Py N-methylpyrrole, β=β-alanine, Dp=dimethylaminopropylamide with AATTAATAT. White balls and diamonds represent pyrrole and β-alanine respectively. Circles with two dots represent lone pairs of electrons on N3 of purines and O2 of pyrimidines at the edges of the bases. Putative bifurcated hydrogen bonds to the amide NH's are illustrated by dashed lines. Panel B: Proposed 1:1 binding model for the complex of P31 (Im-β-Im-Py-β-Im-β-Im-β-Dp where Im N-methylimidazole) with AAGAGAAGAG. Black balls represent imidazole. Circles containing an H represent the N2 hydrogen of guanine. Dashed lines illustrate putative hydrogen bonds. Consensus binding sequences are indicated.

FIG. 7: Specific suppression of the white-mottled eye phenotype with P9

Different polyamides drugs (indicated) were fed to developing w^{m4} flies and representative eye-phenotypes of 5-day old flies are shown. Polyamides (final concentration 100 μM) were present in the fly food from egg laying to hatching. Only oligopyrrole P9, which opens satellite III, was found to suppress the w^{m4} eye phenotype.

FIG. 8: Eye-pigment level determination

Eye-pigments were extracted and determined spectrometrically from 30 carefully dissected fly heads. The pigment levels of 5-day old wm4 male flies of the experiment presented in FIG. (2) are shown. Included are also the eye-pigment levels of heterozygous brown-dominant (bwD/+), white-eye (w67) or wild-type (Canton S.) flies. Genotypes are indicated. Note, that only compound P9 is a suppressor of PEV in wm4 flies, leading to an increased activity of the white gene. Neither P9 nor P31 modify PEV of brown-dominant flies.

FIG. 9: P31 induces homeotic abdomen transformations in bw^{D} flies

Representative eye phenotypes and homeotic transformations are shown. Panels A and B show eye phenotypes of heterozygous brown-dominant (bwD/+) flies raised in the presence of P9, P31 or no drug (indicated). A slight increase in the eye pigmentation (red ommatidia) is observed in P31 treated flies (panel B). This increase is thought to reflect the more advanced age (65-75 hours) of these flies (delayed development) rather than a genuine suppression of PEV. Panel C shows the abdomen of dissected, heterozygous bwD pupae raised in the presence of P31 or P9 (indicated). The A6 to A5 transformation induced by P31 is manifested by the formation of sternite bristles on the A6 segment of flies (arrows). Wild type males are devoid of sternite bristles in A6 (left). Panel D shows the abdomen of dissected pupae raised in the presence of P31 (indicated). The A6 to A5 transformation is more penetrating (Table 1) in the homozygous bwD/bwD flies (arrows). This transformation requires the bwD allele and is not observed in Ubx1/+ flies or other genotypes.

FIG. 10: P31 induces sex comb reduced phenotype in bw^{D} flies

Photographs of the first thoracic male leg showing examples of sex comb phenotypes of heterozygous and homozygous bwD flies obtained after P31 treatment (+P31) compared to untreated flies. The sex comb reduced phenotype is induced by P31 (not P9) only in a bwD genetic background. Mean values for number of teeth are indicated.

FIG. 11: P31 enhances the haltere-to-wing homeotic transformation of Ubx¹ in bw^{D} flies

Photographs of the halteres of animals with different genotypes raised in the presence of P31 or absence (−) Note that P31 enhances the haltere-to-wing transformation of Ubx 1 only in a bwD genetic background. Hence, P31 mimics the genetic interaction of Trl13C and Ubx.

FIG. 12: P31 induces recruitment of GAF to the bwD insert in interphase

Photographs of homozygous bwD polytene nuclei immunostained for GAF (green). DNA is highlighted in blue by DAPI and the bwD insert is highlighted in red by P31T. Black and white inserted panels show the red (right) and green (left) channels separately for the bwD foci. Drug used are indicated. Note that the GAF and P31T signals only overlap following exposure of the permeabilized glands to P31.

### EXAMPLES

### SECTION I: Synthesis and Characterization of DNA Satellite-specific Drugs

Genome projects not only discover a daunting number of new genes, they also yield an enormous amount of non-coding sequence data which must inevitably include ‘architectural’ DNA elements. Architectural DNA is proposed to harbor sequences that mediate nuclear order, chromosome stability and dynamics, sister chromatid cohesion, centromere and telomere formation. While it is conceivable that the tools of proteomics combined with new technologies will eventually allow the assignment of tentative functions to many of the discovered genes (Frederickson, 1999), we are poorly equipped to discover, predict and assign functions to non-genic and architectural DNA. Yet, to understand chromosome biology, we must not only understand gene function but also how these DNA elements impose and then transmit the inheritance of chromosome structural features through cell division.

Biological assays to study architectural DNA are extremely limited. For example, although the phenomena of position effect variation (PEV) is attributed to the positioning of genes near centric heterochromatin (Henikoff, 2000; Karpen, 1994), genetic tools to dissect the functions (if any) of centric satellite DNA are lacking. Is PEV mediated by the sequence satellite repeats, by base composition, by their epigenetic state or simply by the repetitive nature of its chromatin? In view of the difficulties we encounter of assigning functions to large fractions of the genome, we consider the development of new approaches and tools of major importance.

The approach we successfully developed here is based on the synthesis of DNA sequence-specific pseudopeptides (Geierstanger et al., 1994) to study the biological role of architectural DNA involved in chromosome condensation and PEV.

Our interest in these compounds stems from their potential as a tool for molecular and cell biology. Sequence specific minor groove binding drugs may permit a dissection of the role of repeated DNA and of difficult cis-acting elements. Py-Im compounds may be used, if fluorescently labeled, for in situ localization of specific sequences, possibly allowing the study of repeated DNA in living cells.

Here we describe the synthesis and characterization of polyamides that target different DNA satellites and SARs. Interestingly, we observed that compounds targeted to satellite III massively unfold this heterochromatic repeat and that SAR-specific polyamides inhibit chromosome condensation in mitotic Xenopus egg extracts. We also show that pyrrole-imidazole compounds targeted to two different DNA satellites of Drosophila melanogaster have a dramatic effect on PEV and gene expression (Janssen et al., 2000). These observations illustrate the powerful utility of sequence-specific minor groove binding drugs for chromosome research.

### Example 1

### Synthesis of Oligopyrroles for Targeting AT-tracts

To explore the biological potential of polyamides, we aimed at synthesizing compounds that target DNA satellite I, III, V and the interspersed SAR elements. Satellite I (1.672 density) consists of AATAT units encompassing about 6 megabases (Mb) Satellite V (1.705 density) is composed of AAGAG repeats amounting to about 7 Mb (Lohe et al., 1993). Satellite III (1.688 density) has a much longer repeating unit (359 bp) and covers about 10 Mb (Hsieh and Brutlag, 1979). Satellite III repeats behave operationally like SARs (Kas and Laemmli, 1992), the sequence hallmarks of which are numerous clustered AT-tracts. For example, the SAR associated with the Drosophila histone gene cluster is defined by a 656 bp EcoR1/Hinf1 fragment containing 26 AT-tracts of 8 or more Ws (A or T bases) with an average length of 10 base pairs (Gasser and Laemmli, 1986; Mirkovitch et al., 1984). Twenty of these AT-tracts are clustered and separated by a spacer of only a few nucleotides (average 4.5) of mixed base pair sequence.

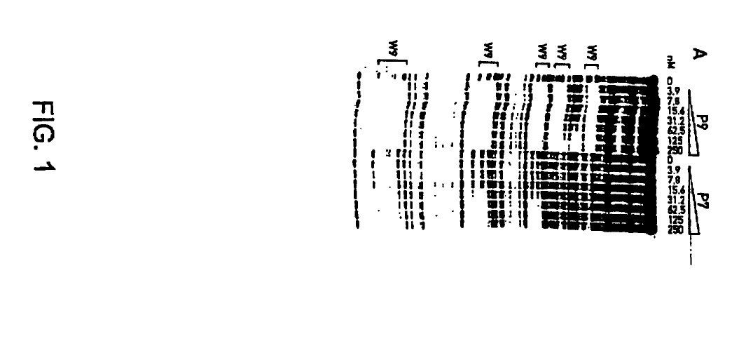

To enlarge binding site size and improve affinity, the number of N-methylpyrrole units can be increased, since each pyrrole carboxamide contacts one AT base pair. However, for compounds containing more than six pyrroles this prediction is no longer valid since the molecule gets out of phase with the base pairs along the minor groove floor. Indeed, the pyrrole-pyrrole distance is about 20% longer then required for perfect match (Goodsell and Dickerson, 1986). In addition, compounds with five or more pyrrole rings are found to be over-bent relative to the pitch of the DNA helix resulting in decreased binding affinities for longer oligopyrroles (de Clairac et al., 1999). To circumvent this mismatch problem, a flexible amino acid (β-alanine) can be introduced in the center of the pyrrole ring system to restore register of the recognition elements and relax the curvature of these crescent-shaped molecules (Youngquist and Dervan, 1987). A pyrrole hexamer termed P9 was synthesized containing a central β-alanine (PyPyPy-β-PyPyPy-β-Dp) and was observed that to bind W9 with 100-fold better affinity (Kapp about 0.75 nM) than P7 (FIG. 1A). This latter value was obtained from footprints that extended to lower ligand concentrations than those shown in FIG. (1A).

### Example 2

### Selective Staining of DNA Satellites and SARs in Nuclei and Polytene Chromosomes

### Drosophila Kc Nuclei

To address the question of the specificity of oligopyrroles when probed on DNA packaged by histones into chromatin the possibility of fluorescently tagging pyrrole ligands in order to stain isolated Kc nuclei and polytene chromosomes for examination by epifluorescence microscopy was explored. If sequence preference is maintained upon tagging and also extends to chromatin, it should be possible to highlight in stained nuclei the positions of the main targets of these fluorescent oligopyrroles (satellites I and III).

Fluorescent groups were coupled to oligopyrroles using commercially available succinimidyl active esters of fluorescein. DNase I footprinting of the fluorescent ligands revealed that these derivatives are differently affected upon tagging. In general, tagging resulted in reduced binding affinity but never affected AT-specificity. Interestingly, for some compounds an improved SAR specificity factor was observed (see Table 1). The SAR specificity of P9F was increased about 4 fold. The fluorescent moiety of this molecule may serve to improve discrimination.

Drosophila Kc nuclei were double stained with ethidium bromide and fluorescein-tagged pyrrole compounds. Ethidium bromide (red) stains nuclear chromatin generally but it also markedly outlines the nucleolus due to the high RNA concentration of this subnuclear domain.

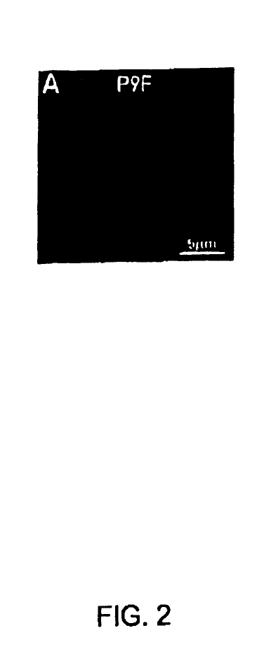

The staining patterns observed with P9F (green) show striking features; the ligand accumulated at one or two subnuclear locations (FIGS. 2A and B) resulting in strong green foci. These foci are generally abutting the nucleolus and are proposed to arise from the expected localization at the abundant AT-rich Drosophila satellites I and III (see below). A low green signal throughout the nucleoplasm is observed with the P9F.

This intense nucleoplasmic localization obtained with the P9F is interpreted to arise from binding to isolated/short AT-tracts that abundantly occur throughout the genome.

### Example 3

### Targeting the GAGAA Repeats of Satellite V with P31

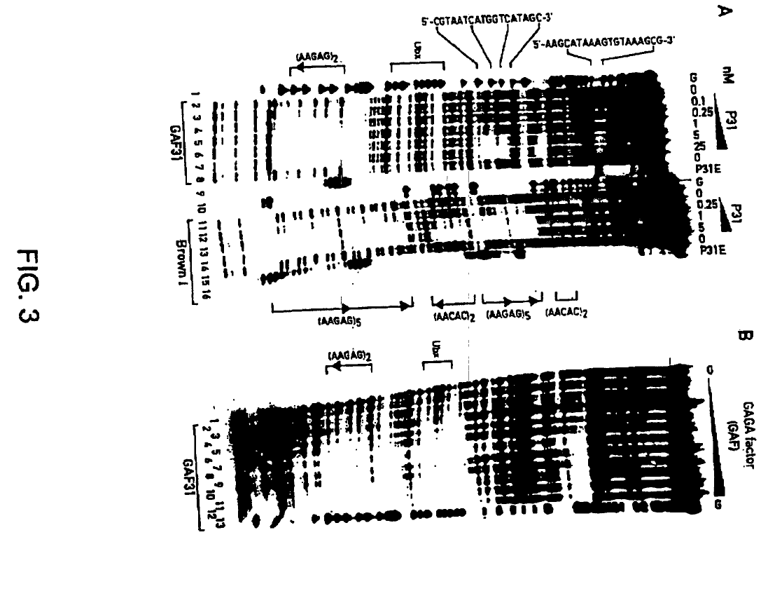

A polyamide that targets the abundant satellite V composed of GAGAA repeats (Lohe et al., 1993) was synthesized. Designing molecules that would bind to this repeat motif represented a challenge since with current knowledge, targeting of sequences containing 5′-GNG-3′ or 5′-GA-3′ with drugs composed of pyrrole and imidazole is difficult. However, successful targeting to sequences containing 5′-GTG-3′ was previously achieved using an Im-β-Im motif where β-alanine replaces the function of pyrrole (Turner et al., 1998). Since β-alanine, like pyrroles, is degenerate for A¹⁰⁸ T and T^{•}A base pairs, we designed a compound based on these observations, to recognize a sequence composed of two tandem GAGAA repeats by systematic placement of -alanine at the N-terminal neighbor of imidazole. The binding affinity and specificity of this compound, termed P31 (=Im-β-Im-Py-β-Im-β-Im-β-Dp), were evaluated by DNAse I footprinting. For this purpose, two different probes were examined, both containing GAGAA repeats. FIG. (3A) shows that P31 binds with subnanomolar affinity to its target binding site, in this case two GAGAA repeats (lanes 2-8). The apparent binding constant of P31 for this sequence was estimated at 0.25 nM. At higher concentrations, protection of two mismatch binding sites was observed. One of these sites contains an AAGTG motif (FIG. 3A).

To determine binding orientation and stoichiometry for P31, we prepared a Fe(II)-EDTA analogue of P31, termed P31E (Im-β-Im-Py-β-Im-β-Im-β-Dp-EDTA). Affinity cleavage was carried out on the footprint probe containing two GAGAA repeats (lane 9) and revealed one major cleavage site flanking the two GAGAA repeats, thereby confirming the assumption chat one P31 molecule binds two GAGAA repeats in a 1:1 drug to DNA complex.

A drawback of this binding model, as opposed to conventional 2:1 drug to DNA complexes, is that P31 is expected to bind degenerate GC and CG base pairs, albeit with different affinity. The consensus sequence can thus be defined as SWSWWSWSWW, where S stands for a G or C and W for A or T. To evaluate binding of P31 to CACAA repeats, we used a second probe that contains two of these repeats as well as five tandem GAGAA repeats. FIG. (3A) shows that P31 protects CACAA repeats with approximately five fold lower affinity than GAGAA repeats (lanes 11-15). Furthermore, affinity cleavage reactions using P31E revealed two major cleavage sites in the GAGAA region (lane 16), showing that in this case, two P31 molecules are bound in tandem to the pentameric GAGAA repeat. Again, it is observed than this molecule binds as a 1:1 drug to DNA complex in an orientation as indicated by arrowheads (FIG. 3A). We propose that special structural features of AT-tracts and GAGAA repeats might favor 1:1 DNA to drug complexes.

In the Examples below it is demonstrated that P31 fed to developing Drosophila melanogaster of the brown-dominant genotype interferes with the function of the GAGA factor (GAF). A footprint experiment was therefore carried out with this protein. The DNA probe (GAF31) used for this purpose contains besides the (AAGAG)₂ motif (the target of P31) a typical promoter proximal GAF binding site derived from the Ubx gene (Biggin et al., 1988). This Ubx site contains the pentameric consensus sequence GAGAG of GAF (Omichinski et al., 1997). The DNase I footprint studies show that, while GAF binds both the (AAGAG)₂ and Ubx motifs, P31 interacts only with the former satellite repeats (compare panels A and B of FIG. 3). Selective Staining of GAGAA Satellite V in Nuclei and Polytene Chromosomes:

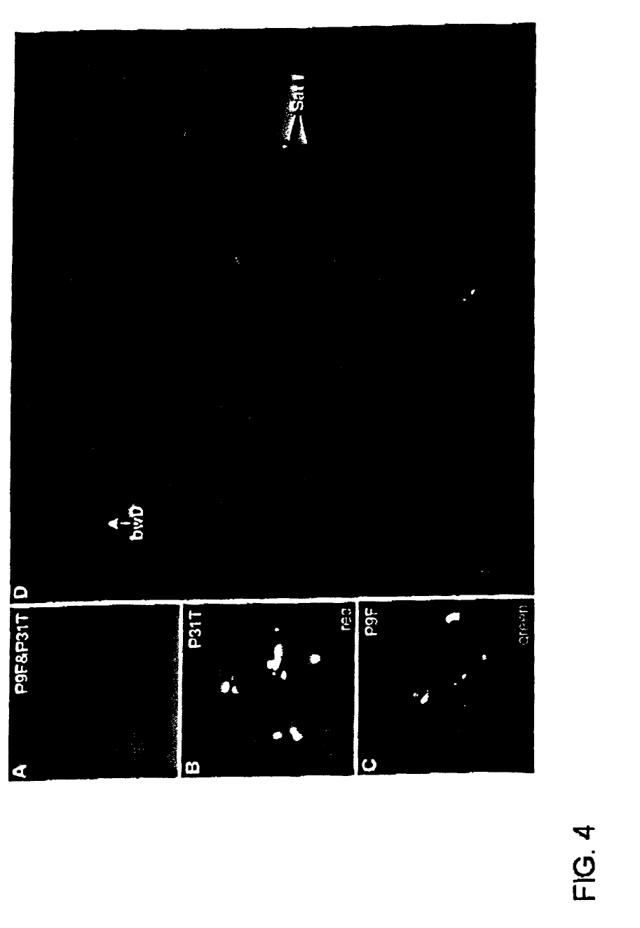

Fluorescent derivatives of P31 were synthesized to visually assess their binding targets by staining of nuclei and chromosomes. DNase I footprinting of the fluorescent ligands revealed that P31T bound the GAGAA sequence with unaltered specificity but with 100 fold reduced binding affinity. Drosophila Kc nuclei were triple stained with DAPI, P9F and P31T and recorded by epifluorescent microscopy. The micrographs obtained again are striking since one notes against the blue DAPI background of nuclear DNA, separate green and red foci stemming from P9F and P31T staining, respectively (FIG. 4A). Closer inspection reveals that these foci are largely non-overlapping (compare panels A and B).

In situ hybridization analysis showed that it is possible to detect satellite I but not satellite V ((GAGAA)n) in polytene chromosomes obtained from wild type flies, supposedly due to a more severe under-replication of satellite V (Platero et al., 1998). Hence, due to this apparent absence of GAGAA repeats, the specificity of P31T for its target binding site cannot be evaluated using ‘normal’ polytene chromosomes. Therefore, to circumvent this limitation, we prepared polytene chromosomes from bown^{dominant} (bw^{D}) flies which harbor an large block of heterochromatin (about 1.7 megabases) composed of GAGAA repeats inserted into the coding region of the brown (bw⁺) gene. This heterochromatic insert appears to be normally polytenized (Csink and Henikoff, 1996; Dernburg et al., 1996; Platero et al., 1998) probably due to its euchromatic localization. Polytene chromosomes were prepared from these flies and stained with P9F, P31T and DAPI. The results obtained were striking (FIG. 4). P31T (red) highlighted conspicuously the bw^{D} GAGAA insert at locus 59E on the right arm of chromosome 2 (2R). No other P31T foci were observed, neither at the chromocenter nor along the euchromatic arms. The familiar band/interband pattern of polytene chromosomes is revealed in blue by DAPI staining.

In summary, we synthesized different satellite-specific polyamides as established by footprinting and epifluorescence microscopy. The Im-Py compound P31 was shown to specifically bind satellite V. All these compounds bind their DNA targets as 1:1 drug to DNA complexes.

### Oligopyrroles Mediate Chromatin Remodelling in a Sequence-specific Fashion

Previously, we reported that exposure of nuclei to distamycin (Py-Py-Py) causes opening of the chromatin fiber, thereby facilitating cleavage by restriction enzymes and topoisomerase II at satellite III (Kas and Laemmli, 1992). Do synthetic polyamides have similar effects on chromatin? As mentioned above, satellite III consists of 359-bp repeats and each repeat unit is packaged in two nucleosomes. Biochemically, satellite III repeats behave as SARs; they preferentially bind nuclear scaffolds, topoisomerase II, HMG-I/Y and MATH20 (Girard et al., 1998; Kas and Laemmli, 1992). Topoisomerase II is also enriched at satellite III in vivo as demonstrated by microinjection of fluorescent topoisomerase II into Drosophila embryos (Marshall et al., 1997). Satellite III contains one prominent topoisomerase II cleavage site per repeat located in every second nucleosomal linker (Kas and Laemmli, 1992). Topoisomerase II cleavage products accumulate in the presence of the cytostatic drug VM26 when Kc nuclei are exposed to Xenopus egg extracts, rich in topoisomerase II. This treatment generates a DNA ladder with a repeat length of 359 bp as revealed by hybridization. The ladder is observed only upon addition of VM26 (FIG. 7A, left). Interestingly, cleavage is massively stimulated by addition P9 (also P7, not shown). Cleavage stimulation is evidenced by an increased intensity of the main repeat band (marked M, one cut per 359-bp repeat) and a shift of the ladder to shorter fragments. Stimulation is maximal at 500 nM and starts to diminish at higher concentrations (FIG. 7A). P9 exposure also results in the appearance of additional, minor bands (marked m) that most likely arise from cleavage within nucleosomes (see discussion). These minor bands are not observed without the drug, even after extended exposure (data not shown).

Next, we tested the potency of P31 in this assay. The results, shown in FIG. (7A), demonstrate that P31 stimulates cleavage considerable less well than P9. That is, while, massive cleavage stimulation is observed with the lowest concentration of P9 (62 nM, FIG. 7A, lane 3) no significant reinforcement of the pattern is observed with P31 up to a concentration of 200 nM (FIG. 7A, lanes 8 to 11). Only at 500 nM is cleavage stimulation by P31 comparable to that obtained with 62 nM of P9 (compare lane 3A to lane 12). Stimulation with P9 is maximal at 500-1000 nM and starts to diminish at higher concentrations. The cleavage ladder induced by P31 at these concentrations is also less pronounced than that of P9 in keeping with the dose response observed. These dosage experiments demonstrate that P9 opens the heterochromatic satellite III at a roughly 10 fold lower concentration than P31.

The data presented above demonstrate that the synthetic oligopyrrole compounds P9 and P7 (not shown) strongly facilitate cleavage by topoisomerase II. The stimulation response to drug treatment is thought to reflect the initial opening of chromatin, that facilitating cleavage.

An additional observation that supports the notion of chromatin opening is that P9 also facilitated cleavage within satellite III by restriction enzymes. Satellite III repeats contain near the topoisomerase II cleavage site a HaeIII restriction sequence. We previously demonstrated that cutting by HaeIII in chromatin (not DNA) is facilitated by distamycin (Kas and Laemmli, 1992). We made a similar observation using P9 (data not shown).

### Discussion of Section I

We explored the potential of sequence-specific minor groove binding polyamides as novel tools to address issues of chromosomal structure, dynamics and the biological functions of non-genic DNA. To this end, we synthesized compounds that interact with satellite I (AATAT), V (GAGAA) and SARs, including the SAR-like satellite III. Although targeting satellite I and SARs can be achieved with ‘conventional’ minor groove binding drugs such as Distamycin, Hoechst and DAPI, their relatively short binding site give rise to high background signals.

Synthesizing compounds that bind GAGAA repeats with high affinity is chemically more challenging since this sequence includes a ‘difficult’ motif. However, impressive targeting to satellite V repeats was obtained with the monomer P31 which is composed of both imidazole and pyrrole units. Structurally, P31 extends recent observations that the ‘difficult’ triplet GWG sequence can be targeted by a Im-β-Im motif where β-alanine is positioned N-terminal of imidazoles (Turner et al., 1998). In P31, this design principal was systematically extended to achieve subnanomolar affinity for two consecutive GAGAA repeats. This design expands the number of sequences that can be targeted, by including GA and GAG motifs.

Pyrrole-Imidazole drugs generally bind the DNA minor groove as antiparallel 2:1 drug to DNA complexes (White et al., 1997). However, the affinity cleavage experiments presented here suggest a 1:1 drug to DNA complex for oligopyrrole P31F. Since binding of two antiparallel oriented molecules requires the expansion of the minor groove (Kielkopf et al., 1998), widening the AT-tract might energetically be too costly. Likewise, crystal structures of B-DNA oligomers demonstrated that GpA steps tend to narrow the minor groove more than GpT steps (Yanagi et al., 1991) which in turn may disfavor 2:1 complexes between P31 and GAGAA repeats.

Epifuorescent microscopy Fluorescent DNA dyes with sequence preference, such as DAPI or Hoechst, are useful, everyday tools of cell biology, medicine and cytogenetics. Sequence specific compounds, if successfully rendered fluorescent, could extend the scientific potential enormously, since innumerable basic questions about chromosome structure, function and dynamics could be addressed using sequence specific dyes. Also, such molecules could facilitate and improve more routine work such as chromosome typing.

Although conjugation of a fluorescent label either at the N- or C-terminal end of oligopyrroles is straightforward, tagging at these positions altered affinity (Table 1).

The main nuclear targets of P31were also demonstrated by staining isolated Kc nuclei and polytene chromosomes with the Texas red derivative, P31T. P31T foci must represent the GAGAA repeats of the centric satellite V (FIG. 4A-C). Positive identification of the main DNA target of P31T was obtained by staining of bw^{D} polytene chromosome whose GAGAA repeat was sharply highlighted by this compound (FIG. 4D). We observed no other P31 signals along the euchromatic arms or at the chromocenter of polytene chromosomes derived from bw^{D} or Canton S. flies. The repetitiveness of these satellite sequences and the polyteny of these chromosomes facilitate the detection of the staining signals. Labeling chromosomes with sequence-specific polyamides is experimentally straightforward, allowing the application of such dyes in innumerable scientific and diagnostic applications. Needless to say, polytene chromosomes might be the ideal object to asses the specificity of sequence-specific hairpin polyamides.

Chromatin opening The chromatin studies revealed that titration of AT-tracts with oligopyrrole P9 massively unfolds the heterochromatic satellite III. Chromatin opening of satellite III is evidenced by the massive stimulation of cleavage by endogenous topoisomerase II when Kc nuclei were exposed to Xenopus egg extracts. We previously made similar, although less pronounced observations, using distamycin and speculated, that unfolding might arise from a displacement of histone H1 or another protein from the nucleosomal linker region (Kas and Laemmli, 1992; Kas et al., 1993). Alternatively, minor groove contacts of the core histones could be of importance for maintaining the heterochromatic state of the chromatin fiber. In contrast to P9, chromatin opening of satellite III required high concentrations of compound P31. In contrast to this, in the accompanying paper, we present data suggesting that, P31 but not P9 can open the heterochromatic GAGAA insert which constitutes the brown-dominant allele (b^{wD}). These observations suggest the DNA minor groove binding polyamides may serve as sequence-specific chromatin openers for silenced genes.

### Materials and Methods

Boc-β-PAM-resin, HBTU, Fmoc-Glu(otBu)-OH, Boc-β-alanine and Boc-γ-aminobutyric acid were purchased from Novabiochem AG, Switzerland. HOBt was from Bachem. The methylester of 4-amino-1-methylpyrrole-2-carboxylic acid hydrochloride was synthesized by Bachem on special request. DMF, acetonitrile (HPLC grade) and 3,3′-diamino-N-methyldipropylamine were purchased from Aldrich. N,N-diisopropylethylamine (DIEA) was from Sigma. Dichloromethane (DCM), thiophenol (PhSH), ethanedithiol (EDT), trifluoroacetic acid (TFA), thiodiglycol, piperidine, N,N′-diisipropylcardodiimide (DIC), dicyclohexylcarbodiimide (DCC) and 3-dimethylamino-1-propylamine were from Fluka. FLUOS (5(6)-carboxyfluorescein-N-hydroxysuccinimide ester) was purchased from Boehringer-Mannheim. All reagents were used without further purification. Glass peptide synthesis reaction vessels (5 ml) with a # 2 sintered glass filter frit were obtained from Verrerie Carouge (Geneva, Switzerland). Analytical and semi-preparatory HPLC was performed as previously described (Baird and Dervan, 1996). Electrospray Ionization mass spectra were obtained in the positive ion mode on a Trio 2000 instrument at the University Medical Center (Geneva, Switzerland).

### Syntheses of Pyrroles for Solid Phase Synthesis

1,2,3-Benzotriazole-1-yl 4-[tert-Butoxycarbonyl)amino]-1-methylpyrrole-2-carboxylate or Boc-Py-Obt was synthesized from 4-amino-1-methylpyrrole-2-carboxylic acid methylester hydrochloride (Baird and Dervan, 1996).

### Manual Solid phase Synthesis of Pyrrole Compounds

Couplings of Boc-Pyrrole were performed as previously described (Baird and Dervan, 1996). Boc deprotections were carried out with 90% TFA, 5% EDT and 5% PhSH (2×30 s, 1×20 min) Cleavage from the resin with 3-dimethylamino-1-propylamine or 3,3′-diamino-N-methyldipropylamine was performed as described (Baird and Dervan, 1996). After cleavage, most of the excess organic base was removed prior to HPLC purification by precipitation of pyrrolic peptides. For this purpose, the reaction mixture was mixed with 3-4 volumes of DCM, followed by the addition of 10 volumes of cold (−20 C.) petroleum ether. The precipitated product was collected by centrifugation and dissolved in 1% TFA to obtain acidic pH.

### Fluorescein-labeling of Compounds

Oligopyrroles with a unique primary amine were obtained by either cleavage of oligopeptides from solid phase with a diamine (3,3′-diamino-N-methyldipropylamine) or deprotection of an N-terminal γ-aminobutyric acid spacer The N-hydroxy succinimide active ester of fluorescein was added in 3 fold excess together with 6 or more equivalents of DIEA. Reactions were allowed to proceed at room temperature for 15 minutes and the fluorescein labeled oligopeptide was purified by HPLC.

### Synthesis of P31 and P31T

P31 (Im-β-Im-Py-β-Im-β-Im-β-Dp) was synthesized in a stepwise fashion by manual solid-phase synthesis from Boc-β-PAM resin as previously described for Imidazole and Pyrrole containing hairpin polyamides (Baird and Dervan, 1996). Since acylation of the imidazole amine on solid phase gives unsatisfactory results, Boc-β-alanine couplings were performed by preparing a Boc-β-Im-OH dimer in solution. The synthesis and activation was as described for dimers of Boc-γ-aminobutyric acid and Imidazole (Baird and Dervan, 1996). For fluorescent labeling of P31, cleavage from the solid support was performed with 3,3′-diamino-N-methyldipropylamine. After HPLC purification, the C-terminal amine was acylated using an commercially available (Molecular Probes) N-hydroxy succinimide active ester of Texas red. The resulting compound was then again purified by HPLC.

### Preparation of Probes for DNase I Footprinting

Synthetic oligonucleotides GATCTAGACGCATATTAATTGCGCTGTCGACGCATTAGTG and GATCCACTAATGCGTCGACAGCGCAATTAATATGCCTCTA were hybridized to obtain the W9 probe, oligomerized by ligation and digested with BamHI and BglII to obtain different tandem repeats. The following oligonucleotides were prepared identically: GAF31 is composed of the oligonucleotides GATCCTCAGAGAGAGCGCAAGAGCGTCCCGGGAGAAGAGAAGAGAGTA and GATCTACTCTCTTCTCTTCTCCCGGGACGCTCTTGCGCTCTCTCTGAG and, BrownI of oligonucleotides GATCCAAGAGAAGAGAAGAGAAGAGAAGAGTACTTATTAACACAACACA and GATCTTGTGTTGTGTTAATAAGTACTCTTCTCTTCTCTTCTCTTCTCTTG. Fragments were purified on low-melt agarose gels and then cloned into a modified pSP64 vector, cut by BamHI and BglII. End-labeling was carried out following digestion with HindIII and a fill-in reaction with Klenow DNA polymerase. The labeled plasmid was cut with PvuII and the target fragments purified from low-melting agarose gels. The 657 bp EcoR1/Hinf1 fragment of the Drosophila histone SAR was cloned into the SmaI site of the modified pSP64 plasmid. This SAR probe was end-labeled following-digestion with EcoR1, then cut with ClaI and the resulting 347 bp fragment purified from low-melting agarose gels.

### DNase I Footprinting

All reactions were performed in a total volume of 40 μl. A polyamide stock solution or buffer (for reference lanes) was added to an assay buffer containing 20 kcpm radiolabeled DNA, affording final concentrations of 10 mM Tris-HCl (pH 7.4), 10 mM KCl, 10 MM MgCl, 5 mM CaCl₂, 0.5 mM EDTA, 0.5 mM EGTA, 1 mM DTT and 0.1% digitonine. The solutions were allowed to equilibrate for at least 2 h at room temperature. Footprinting reactions were initiated by the addition of 2 μl of a DNase stock solution (containing −100 μg DNase I in buffer) and allowed to proceed for 2 min at room temperature. The reactions were stopped by addition of 10 μl of a solution containing 1.25 M NaCl, 100 mM EDTA. Next, 5 μl of a 1% SDS solution was added, followed by 2 μl of a solution containing 1 μg poly(dA-dT), 1 μg salmon sperm DNA and 10 μg glycogen and the DNA was ethanol precipitated (20 min at −20 C.). The reactions were resuspended in 4 μl of 80% formamide loading buffer, denatured 10 min at 85 C., cooled on ice and electrophoresed on 8% polyacrylamide denaturing gels (5% cross-link, 8 M urea) at 30 W for 1 h. The gels were dried and exposed o/n at −70 C.

### Staining of Drosophila Nuclei

Kc Drosophila nuclei were isolated (Mirkovitch et al., 1984), diluted into XBE (10 mM Hepes, pH 7.7, 2 mM MgCl2, 0.1 mM CaCl2, 100 mM KCl, 5 mM EGTA and 50 mM sucrose), fixed with 0.8% fresh paraformaldehyde for 15 minutes and spun onto a round coverslip (10 mm) as described previously (Boy de la Tour and Laemmli, 1988). For washing and staining, coverslips were floated on 60 μl drops of XBE deposited on parafilms. After centrifugation coverslips were washed twice (1 minute), stained for 60 minutes, washed four times (1 minute) and then mounted in PPDI (5 mM Hepes pH 7.8, 100 mM NaCl, 20 mM KCl, 1 mM EGTA, 10 mM Mg SO₄, 2 mM CaCl₂, 78% glycerol, 1 mgr/ml paraphenylene diamine). FIG. (4) panel A was stained with 0.5 μM P9F and 15 μM ethidium bromide (EB).

### Other Methods

Topoisomerase II inhibition and chromosome assembly were as described previously (Girard et al., 1998; Strick and Laemmli, 1995). Affinity cleavage experiments was performed as described elsewhere (Turner et al., 1997).

Section II: Specific Gain and Loss of Function phenotypes induced by Satellite-specific DNA-binding Drugs fed to Drosophila

Position effect variegation (PEV) is an epigenetic gene inactivation phenomenon discovered by Muller (Muller, 1930) arising from chromosomal arrangements that juxtapose euchromatic genes to heterochromatin. Heterochromatin-mediated gene silencing is heritable, epigenetic event that involves no alterations in DNA sequence but instead is due to heritable changes in chromatin structure.

A classical example of PEV is the Drosophila melanogaster allele white-mottled (w^{m4}),which arises from a large inversion that juxtaposes the white gene close to the heterochromatin of the X chromosome. The variegated phenotype of the eyes of w^{m4} flies is noted as red, clonally-derived patches of transcriptionally active cells in an otherwise white colored background where the white gene is silenced (Elgin, 1996; Karpen, 1994; Wakimoto, 1998) A well studied, different case of PEV concerns the brown (bw⁺) gene of Drosophila melanogaster (reviewed by (Henikoff and Comai, 1998). Insertion of a large unit (about 1.7,megabases) of heterochromatic GAGAA satellite repeats into the coding region of this gene causes the brown^{Dominant} (bw^{D}) phenotype (Henikoff et al., 1995). This dominant allele becomes manifested in heterozygous flies (bw^{D}/+), where the heterochromatic insertion inactivates in trans (trans-inactivation) the paired wild type copy of brown (bw⁺). The phenotype of bw⁺/bw^{D} flies is observed as pale brown. variegated eyes due to lack of the pteridine pigment (Henikoff and Dreesen, 1989).

Various molecular models have been discussed to explain PEV. It is often proposed, in the context of white-mottled, that epigenetic inactivation results from spreading of the heterochromatic state along the chromosomes (Locke et al., 1988; Tartof et al., 1989). Other models, largely derived from the brown-dominant studies, suggest that gene silencing is due to local or long-range pairing of heterochromatin and/or altered positioning in the nucleus (Csink and Henikoff, 1996; Csink and Henikoff, 1998; Platero et al., 1998).

Genetic studies in Drosophila melanogaster led to the identification of numerous trans-acting factors implicated in PEV (Elgin and Jackson, 1997). Although these studies allow a better understanding of the biochemistry of heterochromatin, very little is known in higher organism about cis-acting DNA motifs implicated in PEV. It has been suggested that the heterochromatic chromatin state may simply be a consequence of tandem sequence repetition (reviewed by Henikoff, 1998 #97). This suggestion is linked to a phenomenon termed repeat-induced silencing (RIGS) that describes gene silencing caused by tandem gene repetition. RIGS was first described in Arabidopsis thaliana (Assaad et al., 1993) but is also observed in transgene arrays of mice and flies (Dorer and Henikoff, 1994; Dorer and Henikoff, 1997; Garrick et al., 1998).

Eukaryotic genomes contain a vast amount of ‘non-genic’ DNA (e.g. satellite) and PEV provides a rare but very limited experimental opportunity to address the role of this DNA fraction. In view of the difficulties in dissecting and assigning functions to large fractions of the genome, we consider the development of new approaches and tools of major importance. One approach that we successfully applied is the synthesis of artificial DNA sequence specific inhibitors (Girard et al., 1998; Strick and Laemmli, 1995). We were led along this path in attempts to dissect the biological role of scaffold (or matrix) associated regions, called SARs or MARs. These elements are intriguing, since they appear to mediate their biological effect by unusual (‘nonconformist’) mechanisms which may well be representative of the ways non-genic DNA elements implement their functions (Laemmli et al., 1992). The sequence hallmark of SARs are numerous AT-tracts (short sequences of A and T bases) that are generally separated by short, mixed sequence spacers, resulting in clustered AT-tracts. In contrast to standard cis-acting DNA elements, the specific interactions of SARs are not mediated by precise base sequence, but by structural DNA features, such as the narrow minor groove of the AT-tracts and possibly bends (Adachi et al., 1989; Bode et al., 1992; Kas and Laemmli, 1992).

Based largely on techniques developed by Dervan and collaborators (Dervan and Burli, 1999), compounds that target different DNA satellites of Drosophila melanogaster were synthesized and characterized (Janssen et al., 2000). When fed to developing Drosophila melanogaster, it was observed that these satellite-specific drugs can lead to defined gain or loss of function phenotypes. Details are set out below

### Example 4

### Suppression of PEV of White-mottled Flies by Oligopyrrole P9

Two monomeric satellite-specific compounds, termed P9 and P31, were synthesized and characterized as describes in the previous examples. P9 (sequence=PyPyPy-β-PyPyPy-β-Dp where Py=N-methylpyrrole, β=β-alanine and Dp=dimethylaminopropylamide) was found to bind AT-tracts of 9 Ws (A or T bases) with subnanomolar affinity.

We demonstrated further by staining of Kc nuclei and polytene chromosomes using fluorescently tagged P9, that this compound predominantly binds satellite I and III. Satellite I is composed of short AATAT repeats and is therefore a high affinity target for P9. Similarly, due to the numerous AT-tracts in the 359-bp unit of the SAR-like satellite III, specific binding of P9 to this repeat was expected. Compound P31 (sequence=Im-β-Im-Py-β-Im-β-Im-β-Dp where Im=N-methylimidazole) binds two consecutive GAGAA repeats of satellite V. Again, targeting of P31 was confirmed by epifluorescence using a fluorescently labeled compound for staining of nuclei and polytene chromosomes. Both compounds, P9 and P31 were found to bind their targets with subnanomolar affinity in a 1:1 drug to DNA complex by hydrogen bonding schemes as proposed in FIG. (6A, B).

We established further that titration of the AT-tracts of satellite III with P9 resulted in the opening of this heterochromatin block, as revealed by a facilitated cleavage with restriction enzymes or topoisomerase II at internucleosomal linkers (Janssen et al., 2000). Similar observations were made previously with distamycin (Kas and Laemmli, 1992). Furthermore, we observed that P31 was considerably less potent in opening satellite III as compared to P9, presumably due to its low affinity for AT-tracts. In summary, we synthesized two satellite-specific polyamides of similar molecular weight, binding affinity, target site size and interaction mode.

Satellite III is the major component of centric heterochromatin of chromosome X and PEV of white-mottled Drosophila flies is due to an inversion that juxtaposes the white gene (allele w^{m4}) to this centric heterochromatin. Since P9 opens satellite III we asked whether this drug could affect PEV of white-mottled Drosophila flies.

The eye phenotype of w^{m4} flies is quite heterogeneous. About 65% of the eyes are strongly variegated, ranging from quasi-white with little pigment to those containing a generally white background and a number of red patches (defined as the white-mottled class). The remaining flies have eyes with a darker appearance with often larger red patches in an orange background (red-mottled class). Given the phenotypic heterogeneity of w^{m4} flies, we carried out our experiments on a relatively large scale by mixing these compounds directly with semi-synthetic fly medium. Vials were prepared containing a final concentration of either 100 μM P9, P31 or no compound. Equal numbers of 5 to 10 days old w^{m4} flies were allowed to lay eggs for 36 hours. The parents were then removed and progeny development was allowed to proceed at a constant temperature of 18° C. We observed no significant toxicity of P9 and P31 when fed to developing w^{m4} or wild-type flies (Canton S). The timing of the developmental stages was also normal and generally, around 90 to 160 flies hatched per vial in this experiment (Table 2).

For eye phenotype analysis, young flies born on the same day were transferred to a drug-free vial and scored 5 days thereafter. Eye phenotypes were categorized into the two classes defined as white-mottled or red-mottled, after examination under a dissection microscope. This analysis identified P9 as a strong suppressor of PEV. Where in the absence of the drug, 62% of the eyes were scored as white-mottled, only 11% retained this phenotype for the flies raised in the presence of P9. About 90% of the flies had a red-mottled to red eye phenotype upon P9 treatment (FIG. 7, compare A and B, Table 2). In contrast, P31 had no effect on the w^{m4} eye phenotype, since 64% of the flies remained white-mottled (FIG. 7C, Table 2).

To quantify these results further, the red-eye pigments were extracted from 30 heads of males and their relative concentration determined by spectrometry. FIG. (8) shows that the red pigment level corresponding to flies treated with P9 is about 3 times higher than that of the control flies (no drug) or flies fed with P31. Hence, P9 very markedly and specifically suppresses PEV of w^{m4} restoring the red pigment level to about 50% of wild type flies (FIG. 3).

Furthermore, we found that P9 did not suppress PEV of a variegating white reporter transgene inserted at the basis of chromosome 2L (data not shown), indicating that suppression of PEV by P9 is very unlikely the result of a direct interaction of P9 with the promoter of the white gene (see Discussion).

Taken together, these results strongly suggest that suppression of PEV by P9 (not P31) is mediated by specific chromatin opening resulting from titration of AT-tracts which in turn reduces silencing of the rearranged white gene.

### Example 5

### P31 (not P9) Causes a Developmental Delay in Brown-dominant Flies

The P9-induced suppression of the white-mottled phenotype is a remarkable result. It encouraged us to examine a different PEV phenomenon concerning the brown (bw⁺) gene. In contrast to white-mottled, where the proximity of heterochromatin brings about cis-inactivation, PEV mediated by the bw^{D} allele occurs by trans-inactivation (Henikoff and Comai, 1998). In bw^{D}, the brown gene contains an insertion of a large unit of heterochromatic GAGAA satellite repeats in its coding region. This dominant allele becomes manifested in heterozygous flies (bw⁺/bw^{D}) where the heterochromatic insertion is trans-inactivating the paired wild type copy (bw⁺) of brown.

It was demonstrated by footprinting techniques and immunofluorescence the impressive specificity of P31 for GAGAA repeats of bw^{D} (also referred to as bw^{D} repeats). It was therefore of great interest to test whether P31 would affect the eye phenotype of heterozygous bw^{D} flies. Here, P9 serves as the control compound since it does not bind GAGAA repeats. The experiment is similar to that described above for w^{m4}. Vials with semi-synthetic fly food medium were prepared containing a final concentration of 100 μM P31, P9 or no compound. Egg laying was allowed to proceed for 36 hours by homozygous (bw^{D}/bw^{D}) females that were crossed with scarlet (st/st) males. The progeny from this cross is heterozygous for the brown locus and homozygous for scarlet (bw^{D}/+; st/st). In a scarlet background, a modification of the bw^{D} eye color is much easier to observe (Talbert et al., 1994).

In the control experiment, the eye color phenotype of the heterozygous progeny exposed to P9 was pale (light-yellow) and indistinguishable from that of the no-drug control as judged visually or by determination of eye pigment concentration (FIGS. 9A). As observed in the w^{m4} experiment, we noted no toxicity or alteration in the timing of developmental stages upon treatment of bw^{D} flies with P9 (Table 3). This contrasts markedly with the results obtained with P31. This compound not only severely affected the timing of the fly developmental stages, but it also dramatically reduced the viability of the resulting progeny (Table 3). Although a roughly normal timing was noted for the appearance of the second instar larvae, we observed a serious delay of 65-75 hours in both pupation and hatching. Despite this delay, about a normal number of progeny hatched, but these new born flies appeared feeble and often drowned in the fly flood.

Examination of the eye phenotypes of the P31 treated progeny revealed that they contain a larger number of spots with red ommatidia as compared to P9 or untreated flies (FIG. 9A and B). This is also reflected by a slight increase in the eye pigment concentration (FIG. 8). The small, but reproducible, increase in eye pigmentation may reflect their more advanced age (hatching was delayed by 65-75 hours) rather than a genuine increase of bw⁺ function. It is well known that eye pigmentation augments following birth, and e.g. untreated flies of the (bw^{D}/+; st/st) genotype are white at birth but pale yellow (slightly pigmented) at day two. In contrast, P31 treated flies have at birth a level of eye pigmentation that roughly corresponds to that of the controls at day two to three.

In summary, treating heterozygous bw^{D} flies with P31 does not overcome trans-inactivation to a significant extent. The eye phenotype remains pale and strongly variegated. But interestingly, P31 (not P9) induces a serious developmental delay of over 2 days and yields a progeny of very feeble flies, that was not observed upon treatments of Canton S or w^{m4} flies.

The striking dependence of the P31-induced developmental delay on the bw^{D} allele, suggested that a direct interaction between P31 and the GAGAA insert somehow causes a developmental defect(s). Consistent with this idea, we found that the effect of the bw^{D} insert is quantitative, since we found that the effect of P31 on the developmental delay was more severe in homozygous than in heterozygous bw^{D} flies. We observed that about two thirds of the progeny died at the pupal stage and that only a few feeble flies hatched with a delay of about 65 to 75 hours. These experiments established a remarkable molecular interplay between the GAGAA insert of the bw^{D} allele and its target-compound P31.

### Example 6

### In Brown-dominant Flies, P31 Mimics the Trithorax-like Allele Trl^{13C}

Homeotic transformation of A6 to A5: Surprisingly, closer inspection of the P31 progeny revealed a pronounced transformation of the abdominal segment 6 (A6) into A5 (FIG. 9C). This homeotic transformation is manifested by bristles on the ventral A6 which, in contrast to A5, is otherwise bristle-free in males. Bristle induction was found in over 90% of the P31 bw^{D}/+ progeny (Table 3), each displaying between 1 and 10 bristles on A6 (mean=3.75). In contrast, no bristles were observed on A6 in the P9 treated, control males. Importantly, the transformation of A6 to A5 requires both P31 and the bw^{D} genotype, since bristles were never found on segment A6 in flies that are wild type at the brown locus (e.g. w^{m4}, see Table 3).

As mentioned above, bw^{D} homozygosity increased the developmental delay mediated by P31. This observation also extends to the A6 to A5 transformation. While about 10% of heterozygous progeny exposed to P31 lacked bristles on A6, all 40 homozygous bw^{D} males scored had between 3 to 10 bristles (mean value=6, Table 3). Hence, this homeotic transformation and the developmental delay require at least one bw^{D} allele and both have a greater penetration in the bw^{D} homozygous progeny. This observation further reinforces a molecular link between the bw^{D} allele and sensitivity to P31.

A transformation of A6 into A5 is characteristic of certain loss-of-function alleles of the Abd-B gene but is also observed for mutations in genes belonging to the Trithorax-group (Kennison et al., 1998). In particular, the A6 to A5 transformation was previously also observed for the Trl^{13C} allele of the Trithorax-like gene which encodes the GAGA factor (GAF, (Farkas et al., 1994)). Trl^{13C} has a P-element insertion in the first intron of the Trl gene which appears to result in a moderately reduced GAF protein level (Bhat et al., 1996; Farkas et al., 1994). Two presumptive null mutations (Trl^{R67}, Trl^{R85}) of the GAF gene led to lethality at late larval stages (Farkas et al., 1994). It has been suggested that lethality may be late since a large maternal deposition of GAF in the egg allows developmental progression up to larval stages (Bhat et al., 1996).

### Example 7

Sex-comb reduced phenotype: The phenotypic parallels (A6 to A5 transformation) observed between Trl^{13C} flies and bw^{D} flies raised in the presence of P31 suggested that this drug somehow interferes with GAF function. Hence we examined the chemical mimicry by P31 of additional Trl^{13C} phenotypes. Several of the trithorax-Group (trx-G) genes are known to be implicated in the expression of the sex comb reduced (Scr) gene (Kennison et al., 1998). We tested whether the Trl gene might also be involved. For this, we used the Trl^{13C}allele, which occasionally gives rise to viable homozygous flies with no other described additional phenotype (Farkas et al., 1994). We scored homozygous Trl^{13C} males for the number of teeth per sex comb and observed that they were significantly reduced compared to wild-type flies (Table 3). While on average, about 11 teeth per sex comb are found in Canton S. flies and Oregon R., we measured a mean number of 8.6 in homozygous Trl^{13C} flies and also found that about one third had only 7 teeth per sex comb. Furthermore, to test if P31 would also mimic the sex comb reduced phenotype in homo/heterozygous bw^{D} flies, we dissected male legs after treatment of these flies with P31 or P9 (Table 3). Our data show that heterozygous bw^{D} progeny raised in the presence of P31 had mostly 7 to 8 (mean=8.1) sex comb teeth and that this distribution was often shifted to lower numbers, mostly 6 (mean=7.2) in the homozygous case (FIG. 10 and Table 3). Thus, this phenotype depends on the bw^{D} allele and is enhanced by homozygosity. No teeth reduction was observed with w^{m4} flies (Table 3) or other fly stocks (see below) raised in the presence of drugs.

In summary, P31 treatments of bw^{D} flies mimic the A6 to A5 homeotic transformation and the Scr phenotypes of Trl^{13C} mutants. Moreover, homozygous Trl^{13C}/Trl^{13C} flies are known to display a rough-eye phenotype (Farkas et al., 1994), we also observed a slight roughening of the eye after treatment with P31 in bw^{D} progeny (data not shown).

P31 enhances haltere-to-wing transformation in heterozygous bw^{D}/bw; Ubx¹/+ flies: The GAGA factor is know to bind to the promoter of Ubx and to stimulate its transcription in vitro (Biggin and Tjian, 1988) The Trl locus also genetically interacts with Ubx, since Trl^{13C} dominantly enhances the segmental transformation observed in Ubx heterozygotes. That is, flies doubly heterozygous for Trl and Ubx, possess halteres that are further transformed to wing-like structures than Ubx/+ flies (Farkas et al., 1994). We asked, does P31 also chemically mimic the genetic interaction of Ubx and Trl^{13C}? To address this issue, we fed P31 to two genotypes, +/+; Ubx/+ and bw^{D}/bw^{D}; Ubx¹/+. The latter genotype was obtained by crossing bw^{D}/bw^{D}; st/st females and bw^{D}/bw^{D} Ubx¹/Tm3, sb males.

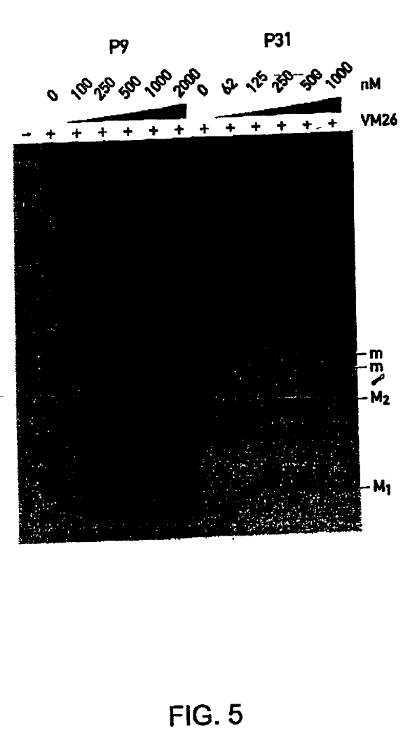

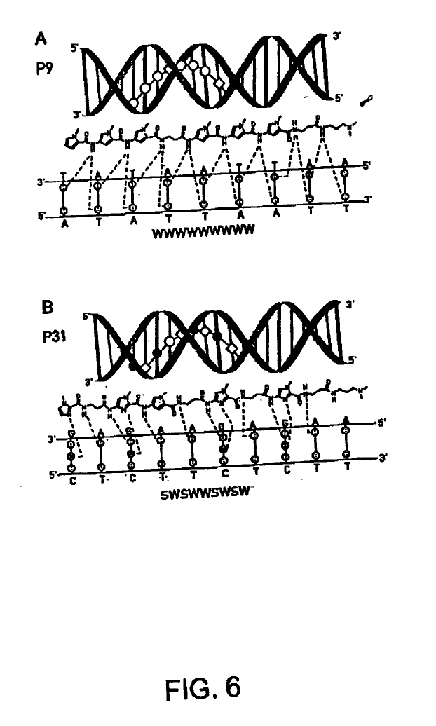

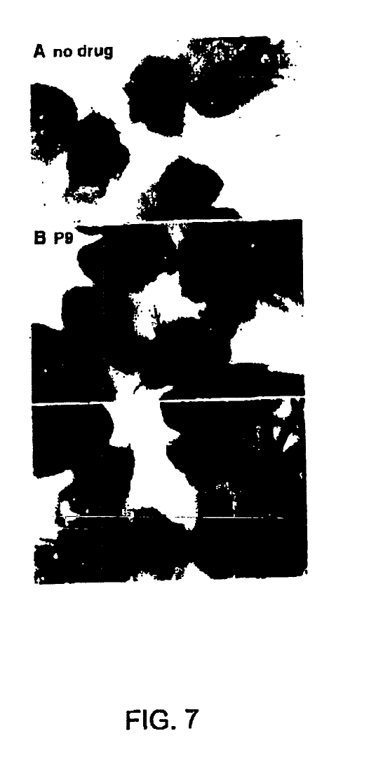

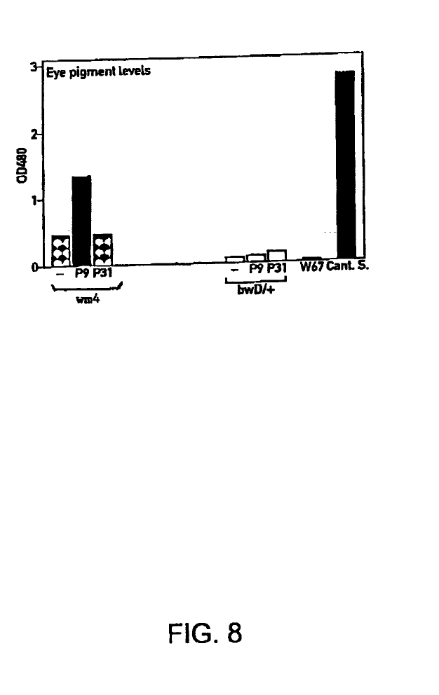

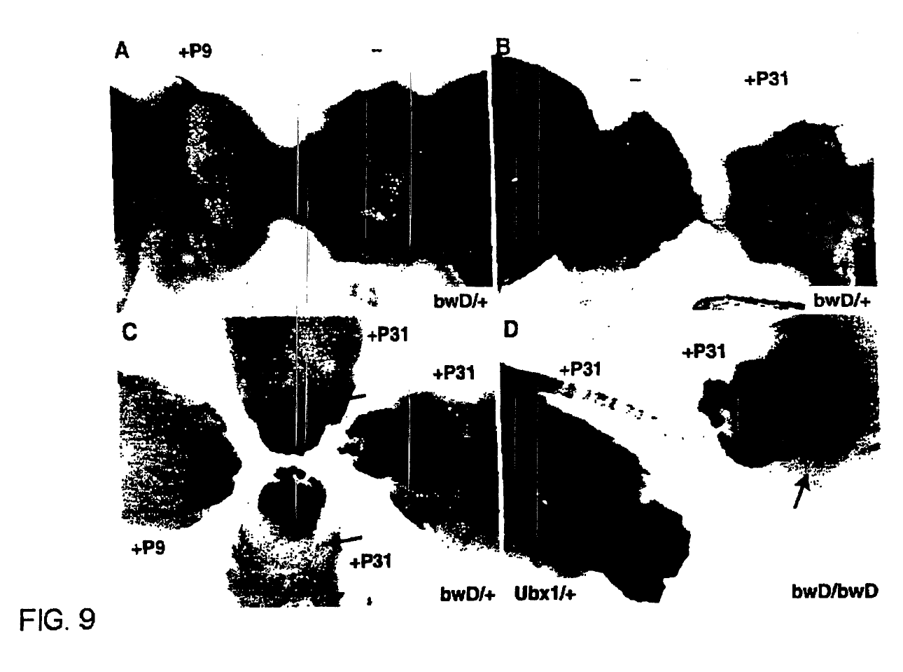

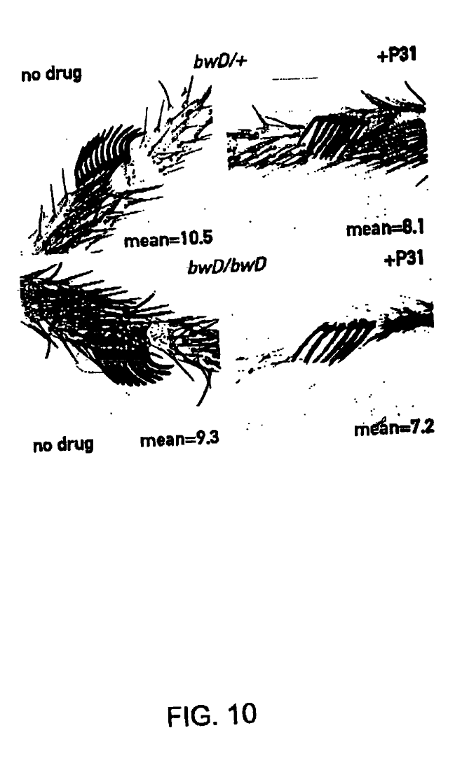

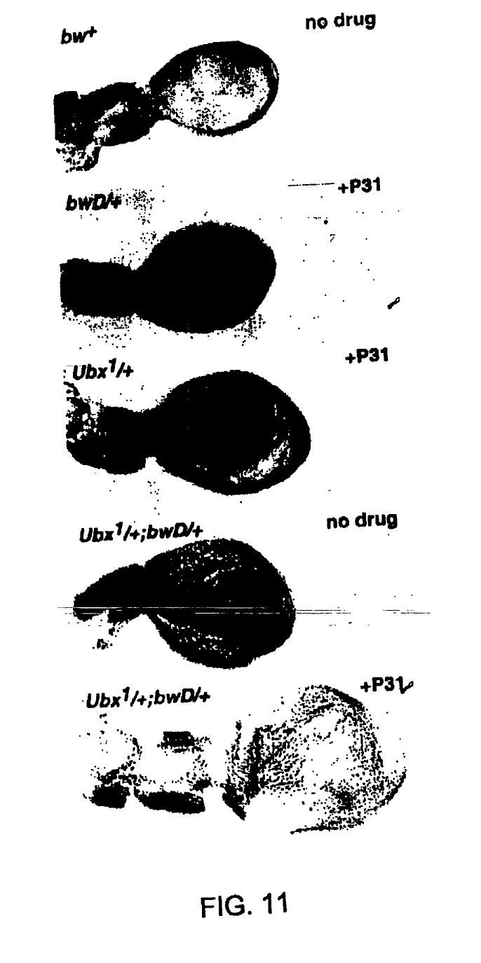

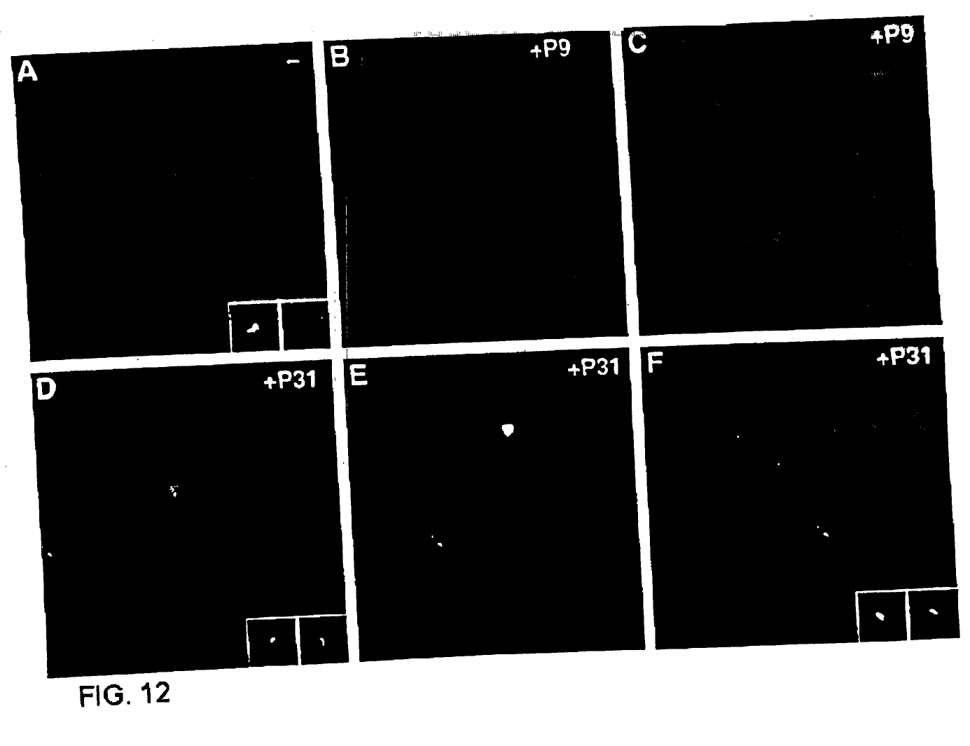

The progeny of these two genotypes raised in the presence of P31 were scored both for the A6 to A5 and the haltere-to-wing transformations as well as for the sex comb reduced phenotypes (Table 3). We observed no altered phenotype in the progeny of Ubx¹/+ flies upon treatment with P31 (FIG. 11C, Table 3). In contrast, severe alterations of the phenotypes were noted in bw^{D}/bw^{D}; Ubx¹/+, flies. P31 blocked their development completely, often before the pupation stage, yielding no newborn flies (Table 3). However, eight of them (out of approximately 100) reached a late pupae stage and thus could be dissected to inspect their morphology. All of these pupae had a strong A6 to A5 homeotic transformation with 2 to 6 bristles (Table 2) and a significant reduction of sex comb teeth (FIG. 5). Furthermore, five of eight pupae that were heterozygous for Ubx¹ and homozygous for bw^{D} (bwD/bwD; Ubx¹/+) displayed clear signs of haltere-to-wing transformations (FIG. 11). This transformation was manifested by an enlarged size and gray color of the halteres, and also by the appearance of numerous bristles at the base of the halteres that are characteristic of the anterior wing margin. Hence, the bw^{D} allele, in combination with the drug P31, enhance the Ubx phenotype. We conclude that the chemical mimicry by P31 in a bw^{D} genetic backgrounds extends to the genetic interaction of Trl^{13C} with Ubx.

### Example 8

### Massive Opening of the GAGAA Satellite Insert with P31

GAF binds GA-rich sequences (Lu et al., 1993). Although there is considerable variability in the binding sequences of GAF, NMR studies identified the pentameric sequence GAGAG as its optimal consensus, where single base pair mutations except the central G have only moderate effects on GAF binding (Omichinski et al., 1997). Recent studies showed that a single trinucleotide repeat GAG is often sufficient to define a specific GAF interaction (Wilkins and Lis, 1998) and that this protein can bind multiple binding sites cooperatively (Katsani et al., 1999)

To test whether P31 binds the GAF consensus sequence (GAGAG), we performed footprinting analysis using a DNA probe that includes, besides 2 consecutive bw^{D} repeats (GAGAAGAGAA), a high affinity binding site for GAF corresponding to the Ubx promoter sequence (Biggin and Tjian, 1988). Inspection of the footprint data showed that, although P31 protects the bw^{D} repeats at a 0.1 nM concentration, no binding is noted at the Ubx GAF site with a ligand concentration up to 25 nM (see FIG. 7A, (Janssen et al., 2000). We also studied the interaction of GAF with this same DNA probe and observed that this protein, in contrast to P31, protects both the bw^{D} repeats and the Ubx site (FIG. 2B, (Janssen et al., 2000))

Database searches indicated that promoter proximal GAF binding sites are normally not composed of bw^{D} repeats, but are either defined by the GAGAG consensus or by multiple GAG motifs. Since these sites are poor targets for P31, it can be concluded that this compound is not likely to interfere directly with gene regulation. The observation that P31 is active only in a bw^{D} genetic background strongly supports this suggestion.

What is then the molecular link between the chemically induced P31 phenotype and the bw^{D} satellite insert? Immunofluorescence studies demonstrated that GAF shuttles during the cell cycle between euchromatin and heterochromatic binding sites. In mitosis, GAF is bound to the heterochromatic AG-rich satellites of metaphase chromosomes (Platero et al., 1998; Raff et al., 1994). In contrast, GAF appears to be actively excluded from the heterochromatic chromocenter of interphase polytene chromosomes but instead is bound to hundreds of sites along the euchromatic arms (Raff et al., 1994; Tsukiyama et al., 1994). The lack of GAF staining at the chromocenter could possibly be due to a detection problem and to the selective under-replication of this heterochromatic satellite (we were unable to detect a bw^{D} satellite signal in the chromocenter with fluorescent P31). But detailed immunofluorescence studies by (Platero et al., 1998) demonstrated that GAF is indeed bound to euchromatin but not to heterochromatin in interphase. In contrast to interphase nuclei, intense GAF staining was observed on heterochromatin GAGAA satellites of mitotic chromosomes. Hence, GAF is clearly heterochromatin bound in mitosis and is then redistributed to numerous euchromatin loci during interphase.

It was demonstrated that P9 opens the heterochromatic satellite III as measured by a massive stimulation of cleavage by topoisomerase II (Janssen et al., 2000). We argued that perhaps P31 may open the bw^{D} insert as to render this heterochromatin more accessible for GAF binding. To address this question, we incubated permeabilized bw^{D} salivary glands either with P31, P9 or no drug and then triply stained the polytene nuclei for GAF (by immunofluorescence), total DNA (with DAPI) and for the bw^{D} insert (with P31T). Stained polytene glands were gently mounted for microscopy without squashing in order examine a large number of nuclei.

FIG. (12) shows polytene nuclei, incubated without compound or with P9 (top row). The thick polytene arms (blue) of intact nuclei are not spread but the euchromatic positions of GAF are observed as sharp, green bands. In red, the position of the bw^{D} insert is highlighted with P31T as single spot. Careful examination revealed that no GAF staining occurs at the position of the P31T signal which marks bw^{D}. This is particularly evident in the black and white inserts since no green GAF signal is overlapping with that of the red P31T (FIG. 12, top row). The staining pattern observed following exposure of salivary glands to P9 is identical to that obtained without drug. These micrographs confirm observations reported by (Platero et al., 1998), who noted no GAF signal over the bw^{D} insert. Evidently, P9 does not alter this pattern.

In contrast, incubation of permeabilized polytene glands with P31 leads to a dramatic GAF redistribution, manifested by a massive co-localization of GAF (green) and P31T (red) (FIG. 7, bottom row). Again, this is best observed in the black and white inserts, where the co-localization of the green GAF and red P31F is evident. Examination of these nuclei further show that GAF staining at euchromatic sites appears considerably weaker; fewer sites are observed and the intensity of the signal is reduced. This observation strongly suggest that P31 (not P9) opens up the heterochromatic bw^{D} satellite to allow binding of GAF resulting in a massive redistribution of GAF from euchromatic sites to heterochromatic sites. It is therefore reasonable to suggest that a similar redistribution occurs in P31 treated bw^{D} flies, where a reduced availability of GAF for euchromatic function leads to a ‘chemical’ gene dosage effect of GAF, as manifested by the observed homeotic transformations.

### Discussion of Section II

The experimental potential of sequence-specific polyamides as tools to better define DNA sequence motifs implicated in PEV has been explored. This epigenetic phenomenon arises from a stochastic gene inactivation either mediated in cis or trans by large blocks of satellite heterochromatin. Two satellite-specific DNA binding drugs were synthesized and fed to developing Drosophila melanogaster flies that display PEV phenotypes. Remarkably, this led to a gain or loss of function, depending on the drug used and the genetic fly background. Most satisfactory is the reciprocity of the experimental observations made. While polyamide drug P9 (not P31) suppressed PEV of white-mottled flies (gain of function), P31 (not P9) mediated homeotic transformations (loss of function) in brown-dominant flies. Both phenomena are in molecular terms explained by chromatin opening of drug-targeted DNA satellites.

Compounds The satellite-specific, DNA minor groove binding drugs used in this study were characterized in detail in Section I. Briefly, compound, P9, is a pyrrole hexamer that targets AT-tracts of 9 (or more) Ws and, P31, is composed of both imidazole and pyrrole units and binds two consecutive GAGAA repeats. Both compounds possess subnanomolar affinity for their DNA target sequence and both were found to bind as 1:1 (drug to DNA) complexes. The main nuclear targets of these compounds where directly revealed using epifluorescent microcopy by staining isolated Kc nuclei and polytene chromosomes with the fluorescent derivatives (P9F and P31T). Both dyes conspicuously marked separate foci in Kc nuclei. P9F targets primarily satellite I and III and P31T targets satellite V which is composed of GAGAA repeats (see FIGS. 4 & 5, (Janssen et al., 2000)).

White-mottled We demonstrated here that feeding oligopyrrole P9 to developing w^{m4} flies significantly suppressed PEV in the resulting progeny. In contrast, P31 and 2 other compounds had no activity on this fly line. This conclusion is based on a statistical and visual analysis of the eye phenotypes and quantitative measurements of the pigment level obtained by extraction of isolated eyes (FIG. 2). This pharmacological experiment was carried out on a relatively large scale, with 90 or more flies per treated progeny, and was found to be highly reproducible.

PEV of white-mottled (w^{m4}) flies arises from a large inversion that juxtaposes the white gene close to the heterochromatin of the X chromosome. The major component of this centric chromatin is satellite III, which operationally behaves like a SAR (scaffold associated region (Kas and Laemmli, 1992)), whose sequence hallmarks are clustered, variably sized, generally long AT-tracts. The 359-bp repeat unit of satellite III contains 10 AT-tracts of 7 or more W bases (average 9.4), accommodated in two phased nucleosomes. We demonstrate that titration of these AT-tracts in vitro with the oligopyrrole P9 unfolds satellite III (Janssen et al., 2000). This is manifested by a strongly facilitated cleavage by topoisomerase II. Cleavage is known to occur in one of the two nucleosomal linker DNA regions (Kas and Laemmli, 1992).

Based on these results, it is reasonable to suggest that P9, when fed to developing flies acts similarly by opening of satellite III and reducing the extend of spreading of the heterochromatic state towards w^{m4}. Our experiments revealed a perfect correlation between chromatin opening in vitro and PEV suppression in flies. P31, which opened satellite III considerably less well, did not suppress PEV of w^{m4} flies. Cis-acting elements involved in PEV have not been identified genetically. The pharmacological studies presented here and our previous MATH20 expression experiment (Girard et al., 1998) offer a novel approach to study this important epigenetic phenomenon. Both series of experiments are strongly congruent, implicating the numerous AT-tracts of satellite III in the establishment of a heterochromatic state and gene silencing at w^{m4}.

Although we favor the possibility that P9 and MATH20 reduce the spreading of heterochromatin toward w^{m4} through long-range effects, we cannot completely rule out the possibility that a local binding site near the white gene (a local SAR) is involved. We attempted to address this issue by studying the effect of P9 on two different variegating miniwhite reporter genes called C3/2L and BL2/Y (Lu et al., 1996; Wallrath and Elgin, 1995). We observed that P9 did not suppress PEV in these stocks (data not shown). This observation demonstrates that suppression of PEV by P9 is not a general phenomenon, but does not rule out the possibility that P9 may act more locally on w^{m4}, since the miniwhite reporter gene, in C3/2L and BL2/Y flies, may lack a nearby P9-responsive element. Nevertheless, we consider a local-acting mode less likely; the enormous target size of satellite III, 11 Mb versus 0.5 kb for a typical SAR, favors a long-range spreading model.

The notion that PEV occurs by spreading of heterochromatin towards w^{m4} stems from morphological studies demonstrating that cis-silencing correlates with a cytological change from a euchromatin to a heterochromatin appearance (Belyaeva et al., 1993; Umbetova et al., 1991). It would therefore be of interest to study whether this cytological change is reversed in flies raised in the presence of P9.

Brown-dominant The brown-dominant phenotype is due to the pairing of homologous chromosomes, one carrying the bw^{D} allele and the other being wild type for brown. Important genetic and cytological experiments by Henifkoff's group led to an attractive model suggesting that the bw^{D} insert mediates an aberrant nuclear interaction with the centric heterochromatin, located on the same chromosome. Hence, according to this model, the bw^{D} insert tethers the paired bw⁺ gene into a heterochromatic environment. This unusual localization, a heterochromatic nuclear compartment, is then proposed to mediate silencing of the bw⁺ gene (Csink and Henikoff, 1996; Talbert et al., 1994)

Our pharmacological experiments showed that feeding P9 or P31 to developing heterozygous bw^{D} flies did not affect their eye phenotype significantly. Our observations discussed below established that feeding P31 to developing bw^{D}/+ or bw^{D}/bw^{D} flies mimics the phenotypic effects of the Trl^{13C} allele of the Trl (GAF) gene. The Trl^{13C} allele is known to enhance (not suppress) the variegated phenotype of w^{m4}, but it does not affect the eye phenotype of bw^{D}/+ flies (Sass and Henikoff, 1998). Hence, since P31 is mimicking the Trl^{13C} mutation (partial loss of GAF function) in developing flies, it is not surprising that P31 does not affect the eye phenotype of bw^{D}/+ flies.

Surprisingly, P31 mediated a developmental delay and several defined homeotic transformations. All these phenotypes depended on the presence of the bw^{D} allele and are dependent on the dosage of bw^{D}. Homozygous Trl^{13C} male flies are known to homeotically transform the abdominal segment A6 to A5. This is observed by the appearance of bristles on A6 (Farkas et al., 1994). This transformation is ‘phenocopied’ by feeding P31 (not P9) to bw^{D} heterozygous flies and enhanced by homozygosity for bw^{D} (FIG. 4, Table 3). The same parallel exists for the sex comb reduced phenotype. In homozygous Trl^{13C}/Trl^{13C} male flies, we counted a reduced number of teeth per sex comb. This establishes a genetic interaction between the Scr and Trl genes. A similar reduction is observed in bw^{D}/+ males raised in the presence of P31 and the number of teeth per sex comb is further reduced in bw^{D}/bw^{D} males (FIG. 10, Table 3). Hence, P31 pharmacologically mimics the Trl^{13C} allele surprisingly well, but this is restricted to bw^{D} flies that carry the heterochromatic GAGAA insert. P31 feeding to other genetic backgrounds never led to these homeotic functions.

The Trl^{13C} carries a P-element insertion in intron 1 of the GAF encoding gene. Detailed studies suggest that this allele is hypomorphic. Therefore, the observed phenotypes arise from a partial Trl loss of function, supposedly by a reduced dose of GAF (Bhat et al., 1996; Farkas et al., 1994). The observed pharmacological phenotypes correspond to loss of function alleles of Abd-B and Scr. The Abd-B gene of the bithorax complex is required for the normal development of abdominal segments A5 through A8. The observation that allele Trl^{13C} and drug P31 affect only segment A6 implicates the cis-acting element iab-6 as a target for GAF (Celniker et al., 1990). Similarly, chromatin immuno-precipitation experiments revealed that GAF is bound to the iab-6 element (Strutt et al., 1997). No molecular information is available concerning the Scr gene, but it is well known that mutations in genes of the Trx-C can lead to a Scr loss of function phenotype.

The strong phenotypic parallels between the effect of P31 and the hypomorphic allele Trl^{13C} suggest, that the pharmacological mimicry by P31 arises from a reduced GAF dose. However, we can practically rule out that P31 primarily acts directly on gene regulatory binding sites in Abd-B and Scr. First, P31 does not interact with typical GAF binding sites (see FIG. 3, (Janssen et al., 2000)) nor is it expected to do so. Secondly, the bw^{D} allele requirement establishes that the P31 effect is molecularly mediated by this heterochromatic insert.

GAF has a very interesting cell-cycle behavior. It binds centric heterochromatin in metaphase and is displaced in interphase to numerous euchromatic sites (Granok et al., 1995; Platero et al., 1999; Raff et al., 1994). Our experiments with permeabilized polytene glands showed that P31 (not P9) mediated a massive redistribution of GAF from the euchromatic binding sites to the heterochromatic bw^{D} insert. The bw^{D} insert of polytene chromosomes is devoid of GAF without drug treatment or exposure to P9 but is highly enriched of GAF in the presence of P31 (FIG. 7). We propose that the bw^{D} insert (and supposedly centric GAGAA repeats) in P31 treated bw^{D} flies, serves as a molecular sink for GAF. This is achieved through chromatin opening mediated by P31. We propose that the inopportune redistribution of GAF to heterochromatin during interphase leads to a depletion of GAF at euchromatic sites In turn, the reduced availability of GAF results in the observed mimicry of the Trl^{13C} allele, that is a partial loss of GAF function. Although our redistribution experiments were not carried out with living fly embryos, but with permeabilized glands, it demonstrates that P31 can interfere with the chromosomal distribution of GAF.

Chromatin opening by P31 of the GAGAA insert could occur long-range, but it was of interest to determine whether P31 and GAF can co-bind the same target sequence. The solution structure of GAF, complexed to its GAGAG consensus sequences revealed a modular binding mode where its single zinc finger and a basic domain (BR2) make contracts in the major groove, while an other basic domain (BR1), wraps around into the minor groove (Omichinski et al., 1997). Although these basic domains are required for a high affinity interaction (Pedone et al., 1996), we observed in competition binding experiments that P31 and GAF can co-bind GAGAA repeats (data not shown).

Is the chromatin opening model quantitatively reasonable? The additional fraction of GAGAA repeats in bw^{D} animals amounts to roughly 17 or 34% for the heterozygous or homozygous genotypes, respectively (Platero et al., 1999). If one assumes that P31 opens the bw^{D} and centric repeats similarly, then the GAF concentration available for euchromatic function would proportionally be reduced by 17 (bw^{D}/bw⁺) or 34% (bw^{D}/bw^{D}) as compared to bw⁺ flies. We consider it reasonable to propose that a fractional reduction to this extent (at euchromatic sites) could affect GAF gene function. Most genes that require GAF for expression, such as Ubx, engrailed and hsp70, contain multiple binding sites and GAF is known to bind such elements highly cooperatively by oligomerization via its POZ domain (Katsani et al., 1999). It is thus conceivable, that a relative small change in GAF concentration could significantly affect the activity of certain genes. If the above mentioned model for chromatin opening and GAF recruitment model is correct, overexpression of GAF should then reduce the effects of P31 in bw^{D} flies. Other molecular scenarios could be envisaged, e.g. centric and bw^{D} heterochromatin could be different biochemically such that P31 only opens the bw^{D} insert. If that were the case, then a much greater relative reduction of the GAF concentration could be achieved in bw^{D} versus bw⁺ flies.

Polyamides were shown to be cell-permeant and to inhibit the expression of targeted genes when added to the media of tissue culture cells (Dickinson et al., 1998; Dickinson et al., 1999; Dickinson et al., 1999; McBryant et al., 1999). Here, we demonstrate that polyamides can affect gene expression of an entire developing organism. In these experiments, flies were raised in the presence of food containing these compounds from egg laying to hatching. Our fly MATH20 expression data showed the suppression of PEV of w^{m4} required its expression around 48 to 72 hours of development when the differentiation of the eye imaginal disc occurs (Girard et al., 1998). It can thus be concluded that these compounds are chemically stable for several days under the experimental conditions. Previous studies also demonstrated that tetracyline added to fly food can also regulate a transactivation system dependent on this antibiotic (Bello et al., 1998). The remarkably unambiguous quality of the fly phenotypes observed, combined with progress in synthesizing sequence-specific polyamides, emphasizes the utility of these chemicals as novel gene tools.

The structure of the heterochromatic fiber is unknown and the experiments presented do not bear on the mechanism whereby heterochromatic satellites are opened by DNA minor groove binding compounds. Such molecules are known to interfere with the binding of proteins that make DNA minor groove contacts (Dorn et al., 1992). Hence chromatin opening might be due to a displacement of heterochromatin-associated proteins, such as HP1, D1 or even the linker histone H1. The N-terminal tails of the core histones are know to make minor groove contracts and it has been speculated that the tail of histone H4 may be involved in mediating the higher-order folding of the chromatin fiber (Luger et al., 1997). DNA minor groove binding drugs could compete for such interactions made by the histone tails and then promote sliding of nucleosomes or unfolding the higher-order chromatin e.g. by disrupting nucleosome-nucleosome interactions. We do not know how long-range spreading of an open chromatin structure is mediated. It has often been discussed that heterochromatinization arises by cooperatively interacting components which ‘polymerize’ along the chromatin fiber where the extent of spreading is governed my mass action. It is easy to see that disruption or displacement of units from this ‘polymer’ will energetically disfavor spreading.

The findings reported here also emphasize that chromatin accessibility may not only be regulated by sophisticated large machines but may be constitutive, that is, determined by the intrinsic property of given chromatin sections to breath (opening/closing) and the general availability of factors that compete by mass action for chromatin opening or closing. It is interesting to speculate that evolution may have positioned of chromatin sections that ‘breath easily’ (e.g. SARs) adjacent to gene regulatory sequences so as to facilitate constitutive accessibility.

Numerous ATP-driven chromatin remodeling complexes have recently been described which facilitate the binding of factors involved in gene expression or, conversely, promote the assembly of repressed heterochromatin (Tyler and Kadonaga, 1999). Such activities are thought to catalyze nucleosome mobility (sliding) or the disruption of nucleosome structure so as to enhance access of DNA-binding factors (experimentally, nucleases) to DNA packaged into chromatin. Chromatin remodeling complexes are very large and composed of several protein subunits. Here we demonstrate that small molecules can serve in flies as heterochromatin remodeling activities. DNA satellites are composed of very large blocks and are therefore relatively easily targeted with sequence-specific compounds. It would be of great interest to explore further whether polyamides can be used as activators of epigenetically silenced genes. Gene silencing is a major problem of most genetic manipulations such as gene therapy, constitutive or regulated expression of genes introduced into plants, animals and microorganism. It might be possible to revert or prevent epigenetic silencing by targeting high affinity polyamides to natural or ‘synthetic’ cis-acting elements of gene expression vectors.

### Experimental Procedures for Section II

### Polyamide Treatment of Flies and Determination of the Eye Phenotypes

Vials for egg laying containing polyamides were prepared as follows: 250 nmoles of P9, P31 was dissolved in 150 μl ddH2O and then mixed with 2.35 g of semi-synthetic fly media pre-heated at 60° C. These vials contained a concentration 100 μM of compound in final. After cooling down to room temperature, ˜300 w^{m4} flies (age 5-10 days) were added to the vial for egg laying. Eggs were collected for a period of 36 hours at 18° C., mother flies were then removed and fly development was allowed to proceed at a constant temperature (18° C.±1). New-born flies of the same day were transferred to a fresh vial and scored after 5 days for eye phenotypes (Girard et al., 1998). For visual inspection of eye-phenotypes (Table 1), both female and male flies were scored. For optical density measurements of the eye-pigments, 30 heads of males were selected, pigments extracted and measured as described (Hazelrigg et al., 1984).

Drug treatments of the bw^{D} flies were performed as for w^{m4} flies except that 30 parental females were allowed to lay eggs. To obtain the heterozygous bw^{D}/+; st/st progeny, 30 virgin bw^{D}/bw^{D}; st/st homozygous females were mated with scarlet homozygous males for about 5 days before transferring them to experimental vials for egg laying.

### Development Delay, Lethality and Homeotic Transformations

The lethality of the progenies was calculated by counting the dead bodies and the total empty pupal shells. The A6 to A5 transformation was detected by bristles that form on abdominal segment A6 on males. All male adult flies obtained (˜50) were scored. Sex combs and halteres were individually inspected and photographed under the microscope (Axiophot, Zeiss) after dissecting at least 30 individuals and incubating them into 30 μl of 0.6 g/ml gum arabic; 4 g/ml chlorohydrate; 35% glycerol under a 24×24 mm coverslip as described (Ashburner, 1989). Since raising double heterozygous flies (Ubx¹/+; bw^{D}/+) led to lethality, we morphologically dissected eight late stage pupae after carefully removing the pupal envelope.

We also found that two males of these progenies had a 90° shift of the genital plate, as previously found in hemizygous bithorax mutants (Karch et al., 1985). Additional mutant phenotypes were occasionally observed for the bw^{D}/+ in P31 treated flies (data not shown). These phenotypes are a missing postvertical bristle on the dorsal head and a triplication of antererior scutellars on segment T2. All of the P31 bw^{D}/bw⁺ progenies had a rough eye phenotype (data not shown).

### Staining of Nuclei and Polytene Chromosomes with Polyamides and Immunofluorescence

Kc nuclei and polytene chromosomes were stained with fluorescent polyamides as described elsewhere (Janssen et al., 2000). For immunofluorescence, polytene glands were carefully dissected from bw^{D} or Canton S. flies in 1× PBS as described (Ashburner, 1989) Whole glands in sol P (1×PBS supplemented with 1 mM MgCl₂ and 0.1% digitonin) were then incubated for 60 minutes with various concentrations of either P31, P9 or no compound at room temperature. P31 (not P9) induced redistribution of GAF during this incubation step. Glands were washed then twice for 5 min with sol P and fixed with fresh paraformaldehyde at a final concentration of 0.8%. Glands were washed 5 times in sol P for 5 min and then blocked for 60 min in the same buffer, supplemented with 5% non-fat milk. Glands were then incubated overnight at 4° C. in a humid chamber with sol P supplemented with 0.5% non-fat milk and the rabbit α-GAGA antibodies kindly provided by Dr. Jordan Raff (Raff et al., 1994). The primary antibodies were removed by washing (5×5 min) and then incubated with the goat anti-rabbit secondary antibodies tagged with FITC (Nordic). Glands were washed as above and stained with 200 nM of P31T to highlight the bw^{D} insert as described (Janssen et al., 2000). Images were recorded with a wide field, deconvolution-type imaging system from DeltaVision.

TABLE 1
Apparent Binding Affinities of Oligopyrroles

| | | Kdₐₚₚ | Kdₐₚₚ | |
|---|---|---|---|---|
| | | W9 | SAR | |
| Compound | Sequence | (nM) | (nM) | Ratio |
| P10 | (Py)₅βDp | 80 | 35 | 2.3 |
| P9 | (Py)₃β(Py)₃βDp | 0.75 | 0.55 | 1.4 |
| P13 | ((Py)₃β)₃Dp | 1.0 | 1.25 | 0.8 |
| P9F | (Py)₃β(Py)₃βD | 4.0 | 1.0 | 4 |
| | * | | | |
| P10F | F*γ(Py)₅βDp | 3000 | 1200 | 2.5 |

γ = 4 amino butyric acid
β = beta alanine
F* = Fluorescein

TABLE 2

| | “White | | |
|---|---|---|---|
| | mottled” | “red Mottled” | |
| | (Strongly | (Weakly | |
| | Variegated) | Variegated) | |
| Compound | (%) | (%) | Total Scored |
| — | 62 | 38 | 219 |
| P31 | 64 | 36 | 91 |
| P9 | 8.9 | 91 | 161 |

### Table 2: Suppression of PEV Eye Phenotype of w^{m4} Flies by P9

New born flies were transferred to fresh vials and scored after five days for eye phenotype. Note that only P9 strongly reduced the fraction of flies that remained strongly variegated (“white mottled”). Suppression of PEV results in the increased activity of the white gene, that is, an augmented level of red pigmentation (“red mottled” as displayed in FIG. 7

TABLE 3

| | | | Pupae delay | | | Sex comb | Haltere to |
|---|---|---|---|---|---|---|---|
| Fly stock | Drug | PEV | (hrs) | Lethality | A6 to A5 | (mean) | Wing |
| W [symbol] | − | − | | 5-10% | 0% | 9-12 (10.6) | − |
| W [symbol] | P9 | +++ | | 5-10% | 0% | 9-12 (10.8) | − |
| W [symbol] | P31 | − | | 5-10% | 0% | 9-13 (10.8) | − |
| bw^{D}/bw⁺ | − | − | | 5-10% | 0% | 9-12 (10.5) | − |
| bw^{D}/bw⁺ | P9 | − | | 5-10% | 0% | nd | − |
| bw^{D}/bw⁺ | P31 | (+?) | 65-67 hrs | 5-10% | 90% (0-10) | 7-10 (8.1) | − |
| bw^{D}/bw⁺ | − | − | | 5-10% | 0% | 8-12 (9.3) | − |
| bw^{D}/bw⁺ | P31 | | 65 hrs-∞ | 66% | 100% (3-10) | 6-9 (7.2) | − |
| Trl^{13C}/Trl^{13C} | − | | | 92% | 87% (0-7) | 7-11 (8.6) | − |
| Ubx¹/+ | − | | | 8% | 0% | 8-12 (10.3) | + |
| Ubx¹/+ | P31 | | | 10% | 0% | 8-12 (10.3) | + |
| Ubx¹/+; bw^{D} | − | | | 7% | 0% | 9-12 (10.0) | + |
| Ubx¹/+; bw^{D} | P31 | | | 100% | 100% (2-6) | 5-9 (7.4) | +++ |

### References

Adachi, Y., Luke, M., and Laemmli, U. K. (1991). Chromosome assembly in vitro: topoisomerase II is required for condensation. Cell 64, 137-48. around naked DNA in vitro. J Cell Biol 115, 1479-89.

Ashburner, M. (1989). “Drosophila”: A Laboratory Manual.” (Cold Spring Harbor, N.Y.: Cold Spring Harbor Laboratory Press).

Assaad, F. F., Tucker, K. L., and Signer, E. R. (1993). Epigenetic repeat-induced gene silencing (RIGS) in Arabidopsis. Plant Mol Biol 22, 1067-85.

Baird, E. E., and Dervan, P. B. (1996). Solid phase synthesis of polyamides containing imidazole and pyrrole amino acids. J Am Chem Soc 118, 6141-6146.

Beerman, T. A., Woynarowski, J. M., Sigmund, R. D., Gawron, L. S., Rao, K. E., and Lown, J. W. (1991). Netropsin and bis-netropsin analogs as inhibitors of the catalytic activity of-mammalian DNA topoisomerase II and topoisomerase cleavable complexes. Biochim Biophys Acta 1090, 52-60.

Bello, B., Resendez-Perez, D., and Gehring, W. J. (1998). Spatial and temporal targeting of gene expression in Drosophila by means of a tetracycline-dependent transactivator system. Development 125, 2193-202.

Belyaeva, E. S., Demakova, O. V., Umbetova, G. H., and Zhimulev, I. F. (1993). Cytogenetic and molecular aspects of position-effect variegation in Drosophila melanogaster. V. Heterochromatin-associated protein HP1 appears in euchromatic chromosomal regions that are inactivated as a result of position-effect variegation. Chromosoma 102, 583-90.

Bhat, K. M., Farkas, G., Karch, F., Gyurkovics, H., Gausz, J., and Schedl, P. (1996). The GAGA factor is required in the early Drosophila embryo not only for transcriptional regulation but also for nuclear division Development 122, 1113-24.

Biggin, M. D., and Tjian, R. (1988). Transcription factors that activate the Ultrabithorax promoter in developmentally staged extracts. Cell 53, 699-711.

Biggin, M. D., Bickel, S., Benson, M., Pirrotta, V., and Tjian, R. (1988). Zeste encodes a sequence-specific transcription factor that activates the Ultrabithorax promoter in vitro. Cell 53, 713-22.

Bode, J., Kohwi, Y., Dickinson, L., Joh, T., Klehr, D., Mielke, C., and Kohwi-Shigematsu, T. (1992). Biological significance of unwinding capability of nuclear matrix-associating DNAs. Science 255, 195-7.

Bode, J.; Stengert-Iber, M., Kay, V., Schlake, T., and Dietz-Pfeilstetter, A. (1996). Scaffold/matrix-attached regions: topological switches with multiple regulatory functions. Crit Rev Eukaryot Gene Expr 6, 115-38.

Boy de la Tour, E., and Laemmli, U. K. (1988). The metaphase scaffold is helically folded: sister chromatids have predominantly opposite helical handedness. Cell 55, 937-44.

Celniker, S. E., Sharma, S., Keelan, D. J., and Lewis, E. B. (1990). The molecular genetics of the bithorax complex of Drosophila: cis-regulation in the Abdominal-B domain. Embo J 9, 4277-86.

Coll, M., Frederick, C. A., Wang, A. H., and Rich, A. (1987). A bifurcated hydrogen-bonded conformation in the d(A.T) base pairs of the DNA dodecamer d(CGCAAATTTGCG) and its complex with distamycin. Proc Natl Acad Sci U S A 84, 8385-9.

Csink, A. K., and Henikoff, S. (1996). Genetic modification of heterochromatic association and nuclear organization in Drosophila. Nature 381, 529-31.

Csink, A. K., and Henikoff, S. (1998). Large-scale chromosomal movements during interphase progression in Drosophila. J Cell Biol 143, 13-22.

de Clairac, R. P. L., Seel, C. J., Geierstanger, B. H., Mrksich, M., Baird, E. E., Dervan, P. B., and Wemmer, D. E. (1999). NMR characterization of the aliphatic b/b pairing for recognition of AT/TA base pairs in the minor groove of DNA. J Am Chem Soc 121, 2956-2964.

Dernburg, A. F., Broman, K. W., Fung, J. C., Marshall, W. F., Philips, J., Agard, D. A., and Sedat, J. W. (1996). Perturbation of nuclear architecture by long-distance chromosome interactions. Cell 85, 745-59.

Dervan, P. B., and Burli, R. W. (1999). Sequence-specific DNA recognition by polyamides. Curr Opin Chem Biol 3, 688-93.

Dickinson, L. A., Gulizia, R. J., Trauger, J. W., Baird, E. E., Mosier, D. E., Gottesfeld, J. M., and Dervan, P. B. (1998). Inhibition of RNA polymerase II transcription in human cells by synthetic DNA-binding ligands [see comments]. Proc Natl Acad Sci U S A 95, 12890-5.

Dickinson, L. A., Trauger, J. W., Baird, E. E., Dervan, P. B., Graves, B. J., and Gottesfeld, J. M. (1999). Inhibition of Ets-1 DNA binding and ternary complex formation between Ets-1, NF-kappaB, and DNA by a designed DNA-binding ligand. J Biol Chem 274, 12765-73.

Dimitrov S, D. M., Wolffe A P (1994). Remodeling sperm chromatin in Xenopus laevis egg

Dorer, D. R., and Henikoff, S. (1994). Expansions of transgene repeats cause heterochromatin formation and gene silencing in Drosophila. Cell 77, 993-1002.

Dorer, D. R., and Henikoff, S. (1997). Transgene repeat arrays interact with distant heterochromatin and cause silencing in cis and trans. Genetics 147, 1181-90.

Dorn, A., Affolter, M., Muller, M., Gehring, W. J., and Leupin, W. (1992). Distamycin-induced inhibition of homeodomain-DNA complexes. Embo J 11, 279-86.

Elgin, S. C. (1996). Heterochromatin and gene regulation in Drosophila. Curr Opin Genet Dev 6, 193-202. Elgin, S. C., and Jackson, S. P. (1997). Chromosomes and expression mechanisms [editorial]. Curr Opin Genet Dev 7, 149-51. extracts: the role of core histone phosphorylation and

Farkas, G., Gausz, J., Galloni, M., Reuter, G., Gyurkovics, H., and Karch, F. (1994). The Trithorax-like gene encodes the Drosophila GAGA factor. Nature 371, 806-8.

Fernandez, L. A., Winkler, M., Forrester, W., Jenuwein, T., and Grosschedl, R. (1998). Nuclear matrix attachment regions confer long-range function upon the immunoglobulin mu enhancer. Cold Spring Harb Symp Quant Biol 63, 515-24. for the Notch gene are in the interband. Cell 54, 461-72.

Forrester, W. C., Fernandez, L. A., and Grosschedl, R. (1999). Nuclear matrix attachment regions antagonize methylation-dependent repression of long-range enhancer-promoter interactions. Genes Dev 13, 3003-3014.

Frederickson, R. (1999). “Functional” proteomics? Nat Biotechnol 17, 1050. Garrick, D., Fiering, S., Martin, D. I., and Whitelaw, E. (1998). Repeat-induced gene silencing in mammals [see comments]. Nat Genet 18, 56-9.

Gasser, S. M., and Laemmli, U. K. (1986). Cohabitation of scaffold binding regions with upstream/enhancer elements of three developmentally regulated genes of D. melanogaster. Cell 46, 521-30.

Geierstanger, B. H., Mrksich, M., Dervan, P. B., and Wemmer, D. E. (1994). Design of a G.C-specific DNA minor groove-binding peptide. Science 266, 646-50.

Girard, F., Bello, B., Laemmli, U. K., and Gehring, W. J. (1998). In vivo analysis of scaffold-associated regions in Drosophila: a synthetic high-affinity SAR binding protein suppresses position effect variegation. Embo J 17, 2079-85.

Goodsell, D., and Dickerson, R. E. (1986). Isohelical analysis of DNA groove-binding drugs. J Med Chem 29, 727-33.

Gottesfeld, J. M., Neely, L., Trauger, J. W., Baird, E. E., and Dervan, P. B. (1997). Regulation of gene expression by small molecules. Nature 387, 202-5.

Granok, H., Leibovitch, B. A., Shaffer, C. D., and Elgin, S. C. (1995). Chromatin. Ga-ga over GAGA factor. Curr Biol 5, 238-41.

Hart, C. M., and Laemmli, U. K. (1998). Facilitation of chromatin dynamics by SARs. Curr Opin Genet Dev 8, 519-25.

Hazelrigg, T., Levis, R., and Rubin, G. M. (1984). Transformation of white locus DNA in drosophila: dosage compensation, zeste interaction, and position effects. Cell 36, 469-81.

Henikoff, S. (2000). Heterochromatin function in complex genomes. Biochim Biophys Acta 1470, 01-08.

Henikoff, S., and Comai, L. (1998). Trans-sensing effects: the ups and downs of being together. Cell 93, 329-32.

Henikoff, S., and Dreesen, T. D. (1989). Trans-inactivation of the Drosophila brown gene: evidence for transcriptional repression and somatic pairing dependence. Proc Natl Acad Sci U S A 86, 6704-8.

Henikoff, S., Jackson, J. M., and Talbert, P. B. (1995). Distance and pairing effects on the brownDominant heterochromatic element in Drosophila. Genetics 140, 1007-17. heterochromatin of Drosophila melanogaster. Genetics 134, 1149-74.

Hirano T, K. R., Hirano M (1997). Condensins, chromosome condensation protein complexes containing XCAP-C, XCAP-E and a Xenopus homolog of

Hirano, T., and Mitchison, T. J. (1991). Cell cycle control of higher-order chromatin assembly the Drosophila Barren protein. Cell 89, 511-21.

Hsieh, T., and Brutlag, D. (1979). Sequence and sequence variation within the 1.688 g/cm3 satellite DNA of Drosophila melanogaster. J Mol Biol 135, 465-81. immunodeficiency virus type 1 integrase. Mol Pharmacol 25 54, 280-90.

Janssen, S., Cuvier, O., Muller, M., and Laemmli, U. K. (2000). Specific Gain and Loss of Function Phenotypes induced by Satellite-specific DNA-binding Drugs fed to Drosophila melanogaster.

Janssen, S., Durussel, T., and Laemmli, U. K. (2000).

Synthesis and Characterization of DNA Satellite-specific Drugs: Chromatin Opening and Chromosome Condensation.

Karch, F., Weiffenbach, B., Peifer, M., Bender, W., Duncan, I., Celniker, S., Crosby, M., and Lewis, E. B. (1985). The abdominal region of the bithorax complex. Cell 43, 81-96.

Karpen, G. H. (1994). Position-effect variegation and the new biology of heterochromatin. Curr Opin Genet Dev 4, 281-91.

Kas, E., and Laemmli, U. K. (1992). In vivo topoisomerase II cleavage of the Drosophila histone and satellite III repeats: DNA sequence and structural characteristics. Embo J 11, 705-16.

Kas, E., Poljak, L., Adachi, Y., and Laemmli, U. K. (1993). A model for chromatin opening: stimulation of topoisomerase II and restriction enzyme cleavage of chromatin by distamycin. Embo J 12, 115-26.

Katsani, K. R., Hajibagheri, M. A., and Verrijzer, C. P. (1999). Co-operative DNA binding by GAGA transcription factor requires the conserved BTB/POZ domain and reorganizes promoter topology. Embo J 18, 698-708.

Kennison, J. A., Vazquez, M., and Brizuela, B. J. (1998). Regulation of the Sex combs reduced gene in Drosophila. Ann N Y Acad Sci 842, 28-35.

Kielkopf, C. L., Baird, E. E., Dervan, P. B., and Rees, D. C. (1998). Structural basis for G.C recognition in the DNA minor groove. Nat Struct Biol 5, 104-9.

Kirillov, A., Kistler, B., Mostoslavsky, R., Cedar, H., Wirth, T., and Bergman, Y. (1996). A role for nuclear NF-kappaB in B-cell-specific demethylation of the Igkappa locus. Nat Genet 13, 435-41.

Laemmli, U. K., Kas, E., Poljak, L., and Adachi, Y. (1992). Scaffold-associated regions: cis-acting determinants of chromatin structural loops and functional domains. Curr Opin Genet Dev 2, 275-85.

Locke, J., Kotarski, M. A., and Tartof, K. D. (1988). Dosage-dependent modifiers of position effect variegation in Drosophila and a mass action model that explains their effect. Genetics 120, 181-98.

Lohe, A. R., Hilliker, A. J., and Roberts, P. A. (1993). Mapping simple repeated DNA sequences in Heterochromatin of Drosophila melanogaster. Genetics, 134,1149-1174

Lu, B. Y., Bishop, C. P., and Eissenberg, J. C. (1996). Developmental timing and tissue specificity of heterochromatin-mediated silencing. Embo J 15, 1323-32.

Lu, Q., Wallrath, L. L., Granok, H., and Elgin, S. C. (1993). (CT)n (GA)n repeats and heat shock elements have distinct roles in chromatin structure and transcriptional activation of the Drosophila hsp26 gene. Mol Cell Biol 13, 2802-14.

Luger, K., Mader, A. W., Richmond, R. K., Sargent, D. F., and Richmond, T. J. (1997). Crystal structure of the nucleosome core particle at 2.8 A resolution [see comments]. Nature 389, 251-60.

Marshall, W. F., Straight, A., Marko, J. F., Swedlow, J., Dernburg, A., Belmont, A., Murray, A. W., Agard, D. A., and Sedat, J. W. (1997). Interphase chromosomes undergo constrained diffusional motion in living cells. Curr Biol 7, 930-9.

Martello, P. A., Bruzik, J. P., deHaseth, P., Youngquist, R. S., and Dervan, P. B. (1989). Specific activation of open complex formation at an Escherichia coli promoter by

McBryant, S. J., Baird, E. E., Trauger, J. W., Dervan, P. B., and Gottesfeld, J. M. (1999). Minor groove DNA-protein contacts upstream of a tRNA gene detected with a synthetic DNA binding ligand. J Mol Biol 286, 973-81.

Miklos, G. L., and Cotsell, J. N. (1990). Chromosome structure at interfaces between major chromatin types:alpha- and beta-heterochromatin. Bioessays 12, 1-6.

Mirkovitch, J., Mirault, M. E., and Laemmli, U. K. (1984). Organization of the higher-order chromatin loop: specific DNA attachment sites on nuclear scaffold. Cell 39, 223-32.

Muller, H. J. (1930). Types of Visible Variations Induced by X-Rays in Drosophila. J. Genet 22, 299-334.

Neamati, N., Mazumder, A., Sunder, S., M., O. J., M., T., Lown, J. W., and Pommier, Y. (1998). Highly potent synthetic polyamides,-bisdistamycins, and lexitropsins as inhibitors of human oligo(N-methylpyrrolecarboxamide)s: effects of peptide length and identification of DNA target

Omichinski, J. G., Pedone, P. V., Felsenfeld, G., Gronenborn, A. M., and Clore, G. M. (1997). The solution structure of a specific GAGA factor-DNA complex reveals a modular binding mode [see comments]. Nat Struct Biol 4, 122-32.

Pedone, P. V., Ghirlando, R., Clore, G. M., Gronenborn, A. M., Felsenfeld, G., and Omichinski, J. G. (1996). The single Cys2-His2 zinc finger domain of the GAGA protein flanked by basic residues is sufficient for high-affinity specific DNA binding [published erratum appears in Proc Natl Acad Sci U S A Jun 25, 1996;93(13):6846]. Proc Natl Acad Sci U S A 93, 2822-6.

Pelton, J. G., and Wemmer, D. E. (1989). Structural characterization of a 2:1 distamycin A.d(CGCAAATTGGC) complex by two-dimensional NMR. Proc Natl Acad Sci U S A 86, 5723-7.

Platero, J. S., Ahmad, K., and Henikoff, S. (1999). A distal heterochromatic block displays centromeric activity when detached from a natural centromere [In Process Citation]. Mol Cell 4, 995-1004.

Platero, J. S., Csink, A. K., Quintanilla, A., and Henikoff, S. (1998). Changes in chromosomal localization of heterochromatin-binding proteins during the cell cycle in Drosophila. J Cell Biol 140, 1297-306.

Poljak, L., Seum, C., Mattioni, T., and Laemmli, U. K. (1994). SARs stimulate but do not confer position independent gene expression. Nucleic Acids Res 22, 4386-94.

Raff, J. W., Kellum, R., and Alberts, B. (1994). The Drosophila GAGA transcription factor is associated with specific regions of heterochromatin throughout the cell cycle. Embo J 13, 5977-83.

Reeves, R., and Nissen, M. S. (1990). The A.T-DNA-binding domain of mammalian high mobility group I chromosomal proteins. A novel peptide motif for recognizing DNA structure. J Biol Chem 265, 8573-82.

Rykowski, M. C., Parmelee, S. J., Agard, D. A., and Sedat, J. W. (1988). Precise determination of the molecular limits of a polytene chromosome band: regulatory sequences

Saitoh, Y., and Laemmli, U. K. (1994). Metaphase chromosome structure: bands arise from a differential folding path of the highly AT-rich scaffold. Cell 76, 609-22.

Sass, G. L., and Henikoff, S. (1998). Comparative analysis of position-effect variegation mutations in Drosophila melanogaster delineates the targets of modifiers. Genetics 148, 733-41. sites. Biochemistry 28, 4455-61.

Spierer, A., and Spierer, P. (1984). Similar level of polyteny in bands and interbands of Drosophila giant chromosomes. Nature 307, 176-8. Strick, R., and Laemmli, U. K. (1995). SARs are cis DNA elements of chromosome dynamics: synthesis of a SAR repressor protein. Cell 83, 1137-48.

Strick, R., and Laemmli, U. K. (1995). SARs are cis DNA elements of chromosome dynamics: synthesis of a SAR repressor protein. Cell 83, 1137-48.

Strutt, H., Cavalli, G., and Paro, R. (1997). Co-localization of Polycomb protein and GAGA factor on regulatory elements responsible for the maintenance of homeotic gene expression. Embo J 16, 3621-32.

Talbert, P. B., LeCiel, C. D., and Henikoff, S. (1994). Modification of the Drosophila heterochromatic mutation brownDominant by linkage alterations. Genetics 136, 559-71.

Tartof, K. D., Bishop, C., Jones, M., Hobbs, C. A., and Locke, J. (1989). Towards an understanding of position effect variegation. Dev Genet 10, 162-76.

Taylor, J. S., Schultz, P. G., and Dervan, P. B. (1984). Sequence specific cleavage of DNA by distamycin-EDTA Fe(II) and EDTA-distamycin. Fe(II). Tetrahedron 40, 457-465.

Tsukiyama, T., Becker, P B., and Wu, C. (1994). ATP-dependent nucleosome disruption at a heat-shock promoter mediated by binding of GAGA transcription factor [see comments]. Nature 367, 525-32.

Turner, J. M., Swalley, S. E., Baird, E. E., and Dervan, P. B. (1998). Aliphatic/aromatic amino acid pairings for polyamide recognition in the minor groove of DNA. J. Am. Chem. Soc. 120, 6219-6226.

Tyler, J. K., and Kadonaga, J. T. (1999). The “dark side” of chromatin remodeling: repressive effects on transcription. Cell 99, 443-6.

Umbetova, G. H., Belyaeva, E. S., Baricheva, E. M., and Zhimulev, I. F. (1991). Cytogenetic and molecular aspects of position effect variegation in Drosophila melanogaster. IV. Underreplication of chromosomal material as a result of gene inactivation. Chromosoma 101, 55-61.

Wakimoto, B. T. (1998). Beyond the nucleosome: epigenetic aspects of position-effect variegation in Drosophila. Cell 93, 321-4.

Wallrath, L. L., and Elgin, S. C. (1995). Position effect variegation in Drosophila is associated with an altered chromatin structure. Genes Dev 9, 1263-77.

Wang, W., and Lown, J. W. (1992). Anti-HIV-1 activity of linked lexitropsins. J Med Chem 35, 2890-7.

White, S., Baird, E. E., and Dervan, P. B. (1997). On the pairing rules for recognition in the minor groove of DNA by pyrrole-imidazole polyamides. Chem Biol 4, 569-78.

Wilkins, R. C., and Lis, J. T. (1998). GAGA factor binding to DNA via a single trinucleotide sequence element. Nucleic Acids Res 26, 2672-8.

Yanagi, K., Privé, G. G., and Dickerson, R. E. (1991). Analysis of local helix geometry in three B-DNA decamers and eight dodecamers. J. Mol. Biol. 217, 201-214.

Youngquist, R. S., and Dervan, P. B. (1985). Sequence-specific recognition of B-DNA by oligo(N-methylpyrrolecarboxamide)s. Proc Natl Acad Sci U S A 82, 2565-9.

Youngquist, R. S., and Dervan, P. B. (1987). A synthetic Peptide binds 16 base pairs of A,T double helical DNA. J Am Chem Soc 109, 564-7566.

1. Process for modulating the function of a DNA element in a eukaryotic cell,
comprising the step of contacting a genomic DNA element, so-called “chromatin responsive element” (CRE),
with a compound having a molecular weight of less than approximately 5 KDa, and having the capacity to bind in a_sequence-specific manner to said CRE,
said step of contacting being carried out in conditions permitting chromatin remodeling of the CRE by said compound,
wherein said chromatin remodeling of the CRE alters the activity of one or more other DNA elements, so called “modulated DNA elements” in the genome.

2. Process according to claim 1 wherein the chromatin remodeling involves altering the epigenetic state of the CRE and/or other DNA elements.

3. Process according to claim 1 wherein the CRE or the other DNA element(s) comprises heterochromatin, heterochromatin-like DNA, euchromatin or naked DNA.

4. Process according to claim 3 wherein the CRE comprises single copy DNA or multicopy DNA

5. Process according to claim 4 wherein the CRE contains identical or non-identical sequence motifs, or functionally interacting multipartite DNA segments.

6. Process according to claim 3 wherein the CRE comprises a DNA element involved in chromosome structure and function.

7. Process according to claim 5 wherein the CRE comprises satellite DNA.

8. Process according to claim 6 wherein the other DNA element comprises a regulatory DNA element.

9. Process according to claim 1, 2 or 3 wherein the CRE is cis-acting with respect to said other DNA element(s), in either a local or long-range manner.

10. Process according to claim 5 wherein the CRE is cis-acting and is contained within said other DNA element.

11. Process according to any one of claims 1 to 4 wherein the CRE is trans-acting in that the other DNA element(s) is or are not directly linked to the CRE.

12. Process according to claim 1 wherein the modulation gives rise to one or more of the following effects: restoration of chromosome function, loss of chromosome function, enhancement of chromosome function, reduction of chromosome function, prevention of chromosome function, modification of the temporal or spatial specificity of gene function, and maintenance of chromosome function.

13. Process according to claim 12 wherein the modulation gives rise to restoration of gene function by suppression of cis or trans epigentic gene silencing.

14. Process according to claim 12 wherein the modulation gives rise to loss of gene function by redistribution, displacement or inhibition of euchromatic binding factors involved in chromosome function, or by allowing the binding of such factors.

15. Process according to any one of claims 1 to 14 wherein the other DNA element(s) is (are) endogenous to said cell.

16. Process according to any one of claims 1 to 14 wherein the other DNA elements(s) is (are) heterologous to said cell.

17. Process according to claim 15 or 16 wherein the CRE is endogenous to said cell.

18. Process according to claim 15 or 16 wherein the CRE is heterologous to said cell.

19. Process according to claim 1 which is carried out in vivo, in vitro or ex vivo.

20. Process according to any one of claims 1 to 19, wherein the sequence-specific DNA binding compound binds to the DNA minor groove.

21. Process according to any one of claims 1 to 20 wherein the sequence-specific DNA binding compound is cell-permeable.

22. Process according to claim 20 or 21 wherein said compound has an apparent binding affinity of at least 5×10⁷ M⁻¹.

23. Process according to claim 22 wherein said compound has an apparent binding affinity of at least 1×10⁹ M⁻¹.

24. Process according to claim 23 wherein said compound has an apparent binding affinity of at least 5×10¹⁰ M⁻¹

25. Process according to claim 1 wherein the sequence-specific DNA binding compound has the capacity to specifically recognise a sequence of at least 6 nucleotides.

26. Process according to claim 20 wherein said compound is an oligomer comprising organic heterocycles.

27. Process according to claim 26 wherein said heterocycles. having at least one annular nitrogen, oxygen or sulphur.

28. Process according to claim 27 wherein said oligomer includes heterocycles chosen from pyrrole, imidazole, triazole, pyrazole, furan, thiazole, thiophene, oxazole, pyridine, or derivatives of any of these compounds wherein the ring NH group is substituted.

29. Process according to claim 28 wherein the heterocyclic oligomer contains N-methylpyrrole (Py) and/or N-methylimidazole (Im).

30. Process according to claim 28 or 29 wherein the heterocyclic oligomer further contains aliphatic amino acids such as β-alanine and γ-aminobutyric acid.

31. Process for modulating the epigenetic state of a heterologous gene in a cell, said process comprising the steps of:
transforming said cell with a nucleic acid sequence comprising said heterologous gene, and with a nucleic acid sequence comprising a so-called heterologous “chromatin responsive element” (CRE),
introducing into said cell a compound which has the capacity to bind in a sequence-specific manner to said heterologous CRE,
said step of contacting being carried out in conditions permitting chromatin remodeling of the heterologous CRE by said compound,
wherein said chromatin modelling of the CRE modulates the epigenetic state of the heterologous gene.

32. Process according to claim 28, wherein the heterologous CRE comprises a sequence whose chromatin status allows the modulation of chromosome function in cis or trans.

33. Process according to claim 31, wherein said cell is eukaryotic.

34. Process according to claim 31, wherein said cell is prokaryotic.

35. Process according to claim 33, wherein said cell is a vertebrate cell, an invertebrate cell, a plant cell.

36. Process according to claim 35, wherein said cell is a mammalian cell, an insect cell, or a yeast cell.

37. Process according to claim 31 wherein the heterologous CRE comprises a SAR-like sequence.

38. Process according to claim 31 wherein the heterologous CRE comprises a GAGAA repeat sequence.

39. Gene expression kit suitable for modulating the epigenetic state of a heterologous gene in a cell, said kit comprising
a nucleic acid molecule comprising said heterologous gene
a nucleic acid molecule comprising a so-called heterologous “CRE”, said heterologous CRE being a sequence whose chromatin status allows the modulation of chromosome function in cis or trans;
a compound having a molecular weight of less than approximately 5 KDa, and having the capacity to bind in a sequence-specific manner to said CRE.

40. Kit according to claim 39 wherein the heterologous CRE comprises a SAR-like AT tract.

41. Kit according to claim 39 wherein the heterologous CRE comprises a GAGAA repeat sequence.

42. Kit according to claim 39 for use in gene therapy.

43. Cell containing a compound having a molecular weight of less than 5 KDa, and having the capacity to bind in a sequence-specific manner to a genomic CRE, said CRE being a sequence whose chromatin status allows the modulation of chromosome function in cis or trans.

44. Cell according to claim 43, wherein said compound specifically binds the DNA-minor groove.

45. Cell according to claim 43 or 44, additionally containing
a nucleic acid molecule comprising a heterologous gene;
a nucleic acid molecule comprising a so-called heterologous “CRE”, said heterologous CRE being a sequence whose chromatin status allows the modulation of chromosome function in cis or trans.

46. Cell according to claim 43 which is a eukaryotic cell.

47. Non-human organism comprising a cell according to claim 43.

48. Organism according to claim 47 which is a non-human animal.

49. Organism according to claim 48 which is a transgenic, non-human animal.

50. Organism according to claim 47 which is a plant.

51. Organism according to claim 50 which is a transgenic plant.

52. Compound having the capacity to bind, in a sequence-specific manner, to a predetermined CRE, said CRE being a sequence whose chromatin status allows modulation of chromosome function in cis or in trans, with the proviso that said compound is not distamycin, HMG-I/Y, or MATH20.

53. Compound having a molecular weight of less than 5 KDa and having the capacity to bind, in a sequence-specific manner, to a predetermined CRE, said CRE being a sequence whose chromatin status allows modulation of chromosome function in cis or in trans, said compound having the capacity to specifically recognise a sequence of at least 6 nucleotides.

54. Compound according to claim 53 having the capacity to specifically recognise a sequence of at least 8 nucleotides.

55. Pharmaceutical composition comprising a compound having the capacity to bind, in a sequence-specific manner, to a predetermined CRE, said CRE being a sequence whose chromatin status allows modulation of chromosome function in cis or in trans, in association with a physiologically acceptable excipient, with the proviso that said compound is not distamycin, HMG-I/Y or MATH20.

56. Pharmaceutical composition comprising a compound having a molecular weight of less than 5 kDa, and having the capacity to bind, in a sequence-specific manner, to a predetermined CRE having at least 6 nucleotides, and said CRE being a DNA sequence whose chromatin status allows modulation of chromosome function in cis or in trans, in association with a physiologically acceptable excipient.

57. Association of pharmaceutical compositions, comprising a first pharmaceutical composition containing
a nucleic acid molecule comprising a heterologous gene
a nucleic acid molecule comprising a so-called heterologous “CRE”, said heterologous CRE being a sequence whose chromatin status allows the modulation of chromosome function in cis or trans, said nucleic acid molecules being in association with a physiologically acceptable excipient, and
a second pharmaceutical composition comprising a compound having the capacity to bind, in a sequence-specific manner, to said CRE, in association with a physiologically acceptable excipient.

58. Association of pharmaceutical compositions according to claim 57, the CRE binding compound in said second pharmaceutical composition has a molecular weight of less than 5 kDa.

59. Composition comprising a compound having the capacity to bind, in a sequence-specific manner, to a predetermined CRE having at least 6 nucleotides, said CRE being a DNA sequence whose chromatin status allows modulation of gene function in cis or in trans, for use in therapy, with the proviso that said compound is not distamycin, HMG-I/Y, or MATH20.

60. Composition comprising a compound having a molecular weight of less than 5 kDa, and having the capacity to bind, in a sequence-specific manner, to a predetermined CRE having at least 6 nucleotides, said CRE being a DNA sequence whose chromatin status allows modulation of gene function in cis or in trans, for use in therapy.

61. Association of compositions according to claim 57 or 58, for use in therapy.

62. Association of compositions according to claim 57 or 58, for use in therapy of genetic disorders resulting from epigenetic status.

63. Use of a compound according to any one of claims 52 to 54 in the preparation of a medicament for the treatment of genetic disorders arising from epigenetic status.

64. Use of an association of compositions according to claim 47 in the preparation of a medicament for the treatment of genetic disorders arising from epigenetic status

65. Use according to claim 63 or 64 wherein the disorder is fragile X syndrome, Prader-Willi syndrome or Wilm's tumour.

66. Use of a kit according to claim 39 for the non-therapeutic modulation of expression of heterologous genes in eukaryotic cells.

67. Use according to claim 66 wherein the modulation is carried out in eukaryotic cells in culture.

68. Use according to claim 66 wherein the modulation is carried out in transgenic animals or in transgenic plants.

69. Compound according to claim 52 to 54 which is fluorescent or fluorescently labelled.

70. DNA-binding compound capable of sequence specific binding to genomic DNA, said compound being an oligomer comprising cyclic heterocycles having at least one annular nitrogen, and optionally at least one aliphatic amino acid residue, wherein said compound is fluorescent or fluorescently labelled.

71. Compound according to claim 69 or 70 wherein the fluorescent label is a fluorescent dye such as fluorescein, dansyl, Texas red, isosulfan blue, ethyl red, malachite green, rhodamine and cyanine dyes.

72. Use of a compound according to claim 69 for probing the epigenetic state and location of DNA in chromosomes and nuclei.

73. Use according to claim 70 for diagnosis of pathological conditions arising from epigenetic status.

74. Use according to claim 73 for pre-symptomatic diagnosis of pathological conditions arising from epigenetic status.

75. Use of a compound according to claim 70 or 71 for chromosome visualisation and marking in diagnosis, forensic studies, affiliation studies, or animal husbandry

76. Method for identifying CREs in a genome, said method comprising:
contacting genomic DNA containing a DNA element whose function is to be modulated, with a series of compounds having the capacity to bind in a sequence specific manner to DNA elements situated upstream, downstream or within the DNA element to be modulated,
selection of those compounds capable of modulating the epigenetic state of the DNA element to be modulated, for example using chromatin probes such as nucleases.
